SEQUENCE LISTING (18 sequences)

SEQ ID NO: 1 - protein, 1004 aa, Oryza sativa
MAGKDEAEGLEARLLLLPPEAAAEEPTRCGGGDGGGGGRKRKKTYLDVLGVCCSAEVALVERLLAPLDGVRVVSVVVASRTVVVEHDPAAAPESAIVKALNKAGLEASVRAYGSSGVVSRWPSPYIVASGVLLTASFFEWLFPPLQCLAVAAVVAGAPPMVRRGFAAASRLSLDINVLMLIAVAGALCLGDYTEAGAIVFLFTTAEWLETLACTKASAGMSSLMGMLPVKAVIATTGEVVSVRDVRVGDVVAVRAGEIVPVDGVVVDGQSEVDERSLTGESFPVPKQPHSEVWAGTMNFDGYIAVRTTALAENSTVAKMERLVEAAQNSRSKTQRLIDSCAKYYTPAVVVVAAGVALIPALLGADGLEQWWKLALVMLVSACPCALVLSTPVASFCAMLRAARMGIFIKGGDVLESLGEIRAVAFDKTGTITRGEFSIDSFHLVGDHKVEMDHLLYWIASIESKSSHPMAAALVEYAQSKSIQPNPENVGDFRIYPGEGIYGEIHGKHIYIGNRRTLARASSPQSTQEMGEMIKGVSIGYVICDGELAGVFSLSDDCRTGAAEAIRELGSLGIKSVMLTGDSSAAATHAQGQLGGVMEELHSELLPEDKVRLVSGLKARFGPTMMVGDGMNDAAALAAADVGVSMGISGSAAAMETSHATLMSSDVLRVPEAVRLGRCARRTIAVNVAGSVAVKAAVLALAAAWRPVLWAAVLADVGTCLLVVLNSMTLLREEWKGGAKEDGACRATARSLVMRSQLAADSQAPNAADAGAAGREQTNGCRCCPKPGMSPEHSVVIDIRADGERQEERPAEAAVVAKCCGGGGGEGIRCGASKKPTATVVVAKCCGGGGGGEGTRCGASKNPATAAVVAKCCSGGGGEGIGCGASKKPTATAVVAKCCGGGGEGTRCAASKKPATAAVVAKCCGGDGGEGTGCGASKRSPPAEGSCSGGEGGTNGVGRCCTSVKRPTCCDMGAAEVSDSSPETAKDCRNGRCCAKTMNSGEVKG

SEQ ID NO: 2 - protein, 951 aa, Oryza sativa
MAGKDEAEGLEARLLLLPPEAAAEEPTRCGGGDGGGGGRKRKKTYLDVLGVCCSAEVALVERLLAPLDGVRVVSVVVASHTVVVEHDPAAAPESVIVKALNKAGLEASVRAYGSSGVVSRWPSPYIVASGVLLTASFFEWLFPPLQCLAVAAVVAGAPPMVRRGFAAASRLSLDINVLMLIAVSGALCLGDYTEAGAIVFLFTTAEWLETLACTKASAGMSSLMGMLPVKAVIATTGEVVSVRDVRVGDVVAVRAGEIVPVDGVVVDGQSEVDERSLTGESFPVPKQPHSEVWAGTMNLDGYIAVRTTALAENSTVAKMERLVEAAQNSRSKMQRLIDSCAKYYTPAVVVVAAGVALIPALLGADGLEQWWKLALVMLVSVCPCALVLSTPVASFCAMLRAARMGIFIKGGDVLESLGEIRAVAFDKTGTITRGEFSIDSFHLVGDHKVEMDHLLYWIASIESKSSHPMAAALVEYAQSKSIQPNPENVADFQIYPGEGIYGEIHGKHIYIGNRRTLARASSPQTIQEMGEMIKGVSIGYVICDGELAGVFSLSDDCRTGAAEAIRELGSLGIKTVMLTGDSSAAATHAQGQLGAVMEELHSELLPEDKVRLVDGLKARFGPTMMVGDGMNDAAALAVADVGVSMGISGSAAAMETSHATLMSSDVLRVPEAVRLGRRARRTIAVNVAGSVAVKAAVLALAAAWRPVLWAAVLADVGTCLLVVLNSMTLLREKWKGGAKEDGACRATARSLAMRSQLAPDSQAPNAAAAAAAGREQTNGCRCCPKPSMSPDHSVVIDIPAGGEHQEERPAAAAVVAKCCGGGGGEGIGCGASKKPTATAVVAKCCGGGGEGTRCGASKRPATAAVVAKCCGGGGGEGTGCGASKRSPPAEGSCSGGEGGINGVGRCGTSVKRPTCCDMGAADVSDSSPETAKDCRNARCCAKTMNSGEVKG

SEQ ID NO: 3 - DNA, 3015 bp, Oryza sativa
atggccggaaaggatgaggcggaagggctcgaggcgaggctgctgctgctgccgcctgaggcggcggcggaggagccgacgaggtgtggcggcggcgacggcggcggcggcgggaggaagcggaagaagacgtaccttgatgtccttggcgtgtgctgctccgcggaggtcgcgctggtggagcggctgctcgcgccgctcgacggcgtccgcgtggtgtccgtcgtcgtggcgtcccgcaccgtcgtcgtcgagcacgaccccgccgccgccccggagtccgccatcgtgaaggcgctgaacaaggccggcctcgaggcgtcggtgcgagcctacggcagcagcggcgtggtcagccggtggccgagcccgtacatcgtcgcctccggcgtcctcctcacggcgtccttcttcgagtggctcttccctcccctgcagtgcctggccgtggcggccgtcgtcgccggcgcgccgccgatggtgcgccgtgggttcgccgcggcgagccggctgtcgctcgacatcaacgtcctcatgctcatcgccgtcgccggcgcgctctgcctcggcgactacacggaggccggcgccatcgtcttcctcttcaccaccgcagaatggctcgagacgctggcctgcacaaaggcgagcgccgggatgtcgtcgttgatgggcatgctgccggtgaaggcggtcatcgcgacgacgggcgaggttgtcagcgtgcgcgacgtccgcgtgggcgacgtcgtcgcggtcagggccggcgagatcgtccccgtcgacggcgtggtggtcgacggccagagcgaggtcgacgagaggagcctcaccggcgagtccttcccggtgccgaagcagccgcactccgaggtctgggccggcacaatgaactttgacggttacattgctgtgagaactacggctctcgccgagaactcgacggtggcgaagatggagaggctggtggaggcggcgcagaacagcaggtcgaagacgcagcggctgatcgattcgtgcgcaaagtactacacgccggccgtggtggttgttgcagcaggagtggccctgatcccggctctgctcggagcagatggccttgagcaatggtggaagctggctctggtgatgcttgtgagcgcgtgcccctgcgcattagtgctgtcgacaccggtggcatccttctgcgcaatgctgcgcgctgcgaggatggggatcttcatcaagggtggagatgttcttgaatcacttggggagatcagggccgtcgcgttcgacaagaccgggacgatcaccagaggagagttcagcatcgactcgttccatctggttggggatcacaaggttgagatggatcatcttctttactggattgcaagcattgagagcaagtcaagccacccaatggcagctgcacttgtggagtatgctcaatccaaatccatccaaccaaacccggaaaatgtgggcgattttcggatatatcccggggaggggatctatggagagatccatggaaagcacatctacattgggaacagaaggacattggcaagagcgtcatcacctcagtcaactcaagaaatgggtgagatgatcaagggcgtgtcgatcggctacgtgatctgcgacggcgagctcgccggcgtcttctcgctctccgacgactgccggaccggcgcggcggaggcgatccgggagcttggatcgctgggcatcaagtcggttatgctgaccggggacagcagcgcggcggcgacgcacgcgcagggccagctcggcggcgtcatggaggagctccactccgagctcctcccggaggacaaggtccggctcgtcagcggcctcaaggcgaggttcggcccgacgatgatggtcggggacgggatgaacgacgcggcggcgctggcggcggcggacgtgggcgtgtccatgggcatctccgggtcggcggcggcgatggagaccagccacgcgacgctcatgtcgagcgacgtgctcagggtccccgaggccgtcaggctcggccggtgcgcccgccggaccatcgccgtcaacgtggccggctcggtggccgtgaaggccgccgtgctcgcgctggccgcggcgtggcgccccgtgctctgggccgccgtgctcgccgacgtcgggacgtgcctcctcgtcgtgctcaacagcatgacgctgctcagggaggagtggaagggcggcgccaaggaggacggcgcgtgccgcgccacggcgaggtcgctggtgatgaggtcccagctcgccgccgactcccaagcacccaacgccgcagacgctggcgccgccggccgtgagcaaacgaacggctgccgttgctgtccgaagccgggcatgtcgcccgagcactcggttgtcatcgacatccgagccgacggcgagcgtcaagaagagcggccggccgaggcggccgtcgttgccaaatgttgcggcggaggcggcggcgagggcatccgctgcggagcctccaagaagccaactgcaacggtcgttgttgccaaatgttgcgggggcggcggcggcggcgagggcaccaggtgcggcgcgtccaagaatccggcaacggcggccgtcgttgccaaatgttgcagcggaggcggcggcgagggcatcggctgcggagcctccaagaagccaactgcaacggccgttgttgccaaatgttgcggcggcggcggcgagggcaccaggtgcgccgcgtccaagaagccggcaacggcggccgtcgttgccaaatgttgcggtggcgatggcggcgagggcaccggatgtggtgcctccaagaggtcgccgccggctgagggaagctgcagcggcggtgaaggcggtaccaatggtgttggtcgttgctgcacgagcgtgaagaggccaacctgttgcgacatgggagcggcggaggtgtccgattcttcgccggagacggcgaaagactgcagaaatgggaggtgttgcgcgaagacgatgaactccggtgaagtgaaaggatga

SEQ ID NO: 4 - DNA, 2856 bp, Oryza sativa
atggccggaaaggatgaggcggaagggctcgaggcgaggctgctgctgctgccgcctgaggcggcggcggaggagccgacgaggtgtggcggcggcgacggcggcggcggcgggaggaagcggaagaagacgtaccttgatgtccttggcgtgtgctgctccgcggaggtcgcgctggtggagcggctgctcgcgccgctcgacggcgtccgcgtggtgtccgtcgtcgtggcgtcccacaccgtcgtcgtcgagcacgaccccgccgccgccccggagtccgtcatcgtgaaggcgctgaacaaggccggcctcgaggcgtcggtgcgagcctacggcagcagcggcgtggtcagccggtggccgagcccgtacatcgtcgcctccggcgtcctcctcacggcgtccttcttcgagtggctcttccctcccctgcagtgcctggccgtggcggccgtcgtcgccggcgcgccgccgatggtgcgccgtgggttcgccgcggcgagccggctgtcgctcgacatcaacgtcctcatgctcatcgccgtctccggcgcgctctgcctcggcgactacacggaggccggcgccatcgtcttcctcttcaccaccgcagaatggctcgagacgctggcctgcacaaaggcgagcgccgggatgtcgtcgttgatgggcatgctgccggtgaaggcggtcatcgcgacgacgggtgaggttgtcagcgtgcgcgacgtccgcgtgggcgacgtcgtcgcggtcagggccggcgagatcgtccccgtcgacggcgtggtggtcgacggccagagcgaggtcgacgagaggagcctcaccggcgagtccttcccggtgccgaagcagccgcactccgaggtctgggccggcacaatgaacttagacggttacattgctgtgagaactacggctctcgccgagaactcgacggtggcgaagatggagaggctggtggaggcggcgcagaacagcaggtcgaagatgcagcggctgatcgattcgtgcgcaaagtactacacgccggccgtggtggttgttgcagcaggagtggccctgatcccggctctgctcggagcagatggccttgagcaatggtggaagctggctctggtgatgcttgtgagcgtgtgcccctgcgcattagtgctgtcgacaccggtggcatccttctgcgcaatgctgcgcgctgcgaggatggggatcttcatcaagggtggagatgttcttgaatcacttggggagatcagggccgtcgcgttcgacaagaccgggacgatcaccagaggagagttcagcatcgactcgttccatctggttggggatcacaaggttgagatggatcatcttctttactggattgcaagcattgagagcaagtcaagccacccaatggcagctgcacttgtggagtatgcccaatccaaatccatccaaccaaacccggaaaatgtcgccgattttcagatatatcctggggaagggatctatggagagatccatggaaagcacatctacattgggaacagaaggacattggcaagagcatcatcacctcagacaattcaagaaatgggggagatgatcaagggcgtgtcgatcggctacgtgatctgcgacggcgagctcgccggcgtcttctcgctctccgacgactgccggactggcgcggcggaggcgatccgggagcttggatcgctgggcatcaagacggtgatgctgaccggggacagcagcgcggcggcgacgcacgcgcagggccagctcggcgccgtcatggaggagctccactccgagctcctcccggaggacaaggtccggctcgtcgacggcctcaaggcgaggttcggaccgacgatgatggtcggcgacgggatgaacgacgcggctgcgctggcggtggcggacgtgggcgtgtccatgggcatctccgggtccgcggcggcgatggagaccagccacgcgacgctcatgtcgagcgacgtgctcagggtccccgaggccgtcaggctcggccggcgcgcccgccggaccatcgccgtcaacgtggcgggctcggtggccgtgaaggccgccgtgctcgcgctcgccgcggcgtggcgccccgtgctctgggccgccgtgctcgccgacgtcgggacgtgcctcctcgtcgtgctcaacagcatgacgctactgagggagaagtggaagggcggcgccaaggaggacggcgcatgccgcgccacggcgaggtcgctggcgatgaggtcccagctcgcccccgactcccaagcgcccaacgccgcagccgctgccgccgccggccgtgagcaaacgaacggctgccgttgctgtccgaagccgagcatgtcgcccgaccactcggttgtcatcgacatccctgccggcggcgagcatcaagaagagcggccggccgcggcggccgtcgttgcgaaatgttgtggcggaggcggcggtgagggcatcggctgcggagcctcgaagaagccaactgcaacggccgttgttgccaaatgttgcggcgggggcggcgagggcaccaggtgcggcgcgtccaagaggccggcaacggcggccgtcgttgccaaatgttgcggtggcggtggcggcgagggcaccggatgtggtgcctccaagaggtcgccgccggctgagggaagctgcagcggcggtgaaggcggcatcaacggtgttggtcgttgcggcacgagcgtgaagaggccaacctgttgcgacatgggagcggcggatgtgtccgattcttcgccggagacggcgaaagactgcagaaatgcgaggtgttgcgcgaagacgatgaactccggtgaagtgaaaggatga

SEQ ID NO: 5 - DNA, 32 bp, Artificial
  Primer
atctagaagcataaaagaatagagccgtggac

SEQ ID NO: 6 - DNA, 22 bp, Artificial
  Primer
ggatgcgtcaatcagtttacca

SEQ ID NO: 7 - DNA, 21 bp, Artificial
  Primer
ggcacaatgaactttgacggt

SEQ ID NO: 8 - DNA, 25 bp, Artificial
  Primer
ctcttctggacaagcttccttaatc

SEQ ID NO: 9 - DNA, 26 bp, Artificial
  Primer
aggatccatggccggaaaggatgagg

SEQ ID NO: 10 - DNA, 31 bp, Artificial
  Primer
tggatccgcaacatcatcctttcacttcacc

SEQ ID NO: 11 - DNA, 22 bp, Artificial
  Primer
tccatccaaccaaacccggaaa

SEQ ID NO: 12 - DNA, 22 bp, Artificial
  Primer
tgccaatgtccttctgttccca

SEQ ID NO: 13 - DNA, 23 bp, Artificial
  Primer
ggtcaacttgttgattcccctct

SEQ ID NO: 14 - DNA, 21 bp, Artificial
  Primer
aaccgcaaaatccaaagaacg

SEQ ID NO: 15 - DNA, 32 bp, Artificial; identical to SEQ ID NO: 5
  Primer

SEQ ID NO: 16 - DNA, 27 bp, Artificial
  Primer
atctagaatgcaagtggggatcaagga

SEQ ID NO: 17 - DNA, 27 bp, Artificial
  Primer
atccggaatggccggaaaggatgaggc

SEQ ID NO: 18 - DNA, 26 bp, Artificial
  Primer
ttccggatcctttcacttcaccggag

(19) United States
(12) Patent Application Publication
(10) Pub. No.: US 2012/0174257 A1
(43) Pub. Date: Jul. 5, 2012

(54) USE OF GENE INVOLVED IN ACCUMULATION OF CADMIUM IN PLANTS

(75) Inventor: Jian Feng Ma, Okayama (JP)

(73) Assignee: NATIONAL UNIVERSITY CORPORATION OKAYAMA UNIVERSITY, Okayama (JP)

(21) Appl. No.: 13/394,569

(22) PCT Filed: Sep. 3, 2010
(86) PCT No.: PCT/JP2010/065173

§ 371 Date: Mar. 7, 2012

(30) Foreign Application Priority Data
Sep. 9, 2009 (JP) 2009-208541

(51) Int. Cl. A01H 1/06; C12N 15/29; A01H 5/00

(52) U.S. Cl. 800/278; 800/298; 536/23.6

(57) ABSTRACT

Identifying a gene responsible for the accumulation of cadmium in plants, the present invention provides a method for using such a gene. A polynucleotide encoding a polypeptide consisting of the amino acid sequence of SEQ ID NO: 1, a polynucleotide encoding a polypeptide in which one or several amino acids are substituted, deleted, and/or added in the amino acid sequence of SEQ ID NO: 1 and which has activity to change the localization of cadmium accumulation, a polynucleotide encoding a polypeptide consisting of the amino acid sequence of SEQ ID NO: 2, or a polynucleotide encoding a polypeptide in which one or several amino acids are substituted, deleted, and/or added in the amino acid sequence of SEQ ID NO: 2 and which has activity to change the localization of cadmium accumulation is introduced into a plant such that the polynucleotide is able to be expressed.

### TECHNICAL FIELD

The present invention relates to use of a gene responsible for cadmium accumulation in plants.

### BACKGROUND ART

Cadmium, known as a substance that causes Itai-itai disease, is a highly toxic heavy metal. Since cadmium is a heavy metal present naturally in minerals, soils, etc., and is present with metals such as silver, copper, and zinc, cadmium is discharged into the environment and accumulated in soils in association with mining and refining of such metals.

Cadmium is taken into human bodies through various agricultural products, such as vegetables and cereals, produced on soils contaminated with cadmium, and has adverse effects on human health. Therefore, for control of the intake of cadmium from agricultural products, the Food Hygiene Law of Japan stipulates standard levels of cadmium concentration in agricultural products. For example, according to the law, rice whose cadmium concentration in brown rice is not less than 0.4 ppm and less than 1.0 ppm shall not be sold as food. Rice whose cadmium concentration is not less than 1 ppm shall not be sold for any purposes or used for processing etc., and is incinerated in actual practice.

For the purpose of reducing cadmium concentrations in agricultural products such as rice to levels less than or equal to the standard levels and thereby ensure the safety of food, attempts to clean up cadmium-contaminated soils have been made through conventional techniques such as soil washing, earth filling, and soil dressing (e.g., see Patent Literature 1 and Non-patent Literature 1).

### CITATION LIST

Patent Literature 1
International Publication No. 2004/037453 Pamphlet (Publication Date: May 6, 2004)

Non-Patent Literature 1
Calmano W, Mangold S, Stichnothe H, Thoming J: Clean-up and assessment of metal contaminated soils; in Treatment of Contaminated soil, ed. R. Stegmann, G. Brunner, W. Calmano and G. Matz, p. 471-490, Springer, Berlin (2001)

### SUMMARY OF INVENTION

### Technical Problem

However, such conventional methods as soil washing, earth filling, and soil dressing have drawbacks: high cost, difficulty in obtaining uncontaminated soils for use in earth filling and soil dressing, etc. Further, such conventional methods entail chemical or physical treatment of soils and hence may pose a risk of disrupting the ecosystem. Under such circumstances, cadmium-contaminated agricultural lands are abandoned in disuse.

From a point of view of efficient use of cadmium-contaminated agricultural lands, it is considered to be useful to achieve plants in which cadmium contained in soil is hardly accumulated. However, it has been unknown what gene is responsible for cadmium accumulation in plants.

The present invention has been made in view of the foregoing problems, and it is an object of the present invention to identify a gene responsible for cadmium accumulation in plants and provide a use for such a gene.

### Solution to Problem

The inventor of the present invention diligently studied to solve the foregoing problems. In the result, the inventor of the present invention became the first person to find that OsHMA3 gene, which resides on chromosome 7 in rice, is responsible for cadmium transportation in plants, and the inventor of the present invention has thus accomplished the present invention. That is, the present invention encompasses the following inventions:

A method according to the present invention for producing a transgenic plant is a method for producing a transgenic plant changed in localization of cadmium accumulation, comprising the step of introducing a polynucleotide into a plant such that the polynucleotide is able to be expressed, the polynucleotide being: (a) a polynucleotide encoding a polypeptide consisting of the amino acid sequence of SEQ ID NO: 1; (b) a polynucleotide encoding a polypeptide in which one or several amino acids are substituted, deleted, and/or added in the amino acid sequence of SEQ ID NO: 1 and which has activity to change the localization of cadmium accumulation; (c) a polynucleotide encoding a polypeptide consisting of the amino acid sequence of SEQ ID NO: 2; or (d) a polynucleotide encoding a polypeptide in which one or several amino acids are substituted, deleted, and/or added in the amino acid sequence of SEQ ID NO: 2 and which has activity to change the localization of cadmium accumulation.

A transgenic plant according to the present invention is produced by the method according to the present invention for producing a transgenic plant.

A kit according to the present invention is a kit for producing a transgenic plant changed in localization of cadmium accumulation, comprising a polynucleotide, the polynucleotide being: (a) a polynucleotide encoding a polypeptide consisting of the amino acid sequence of SEQ ID NO: 1; (b) a polynucleotide encoding a polypeptide in which one or several amino acid are substituted, deleted, and/or added in the amino acid sequence of SEQ ID NO: 1 and which has activity to change the localization of cadmium accumulation; (c) a polynucleotide encoding a polypeptide consisting of the amino acid sequence of SEQ ID NO: 2; or (d) a polynucleotide encoding a polypeptide in which one or several amino acids are substituted, deleted, and/or added in the amino acid sequence of SEQ ID NO: 2 and which has activity to change the localization of cadmium accumulation.

Additional objects, features, and strengths of the present invention will be made clear by the description below. Further, the advantages of the present invention will be evident from the following explanation in reference to the drawings.

### Advantageous Effects of Invention

The method according to the present invention for producing a transgenic plant brings about an effect of making it possible to produce a transgenic plant changed in localization of cadmium accumulation, or, specifically, making it possible to produce a transgenic plant changed in localization of cadmium accumulation such that cadmium accumulation is more localized in the roots as compared with a wild-type plant or changed in localization of cadmium accumulation such that cadmium accumulation is more localized in those parts of the plant except for the roots as compared with the wild-type plant.

The transgenic plant with such a change in localization of cadmium accumulation that cadmium accumulation is more localized in the roots makes it difficult for cadmium to be accumulated in those parts of the plant except for the roots. With this feature, the transgenic plant according to the present invention can be cultivated even in a cadmium-containing soil. Further, by being cultivated in a cadmium-containing soil, the transgenic plant with such a change in localization of cadmium accumulation that cadmium accumulation is more localized in those parts of the transgenic plant except for the roots can be suitably used as a soil-cleansing plant that removes cadmium from the soil easily at low cost.

Further, use of the kit according to the present invention makes it possible to easily produce a transgenic plant changed in localization of cadmium accumulation.

### BRIEF DESCRIPTION OF DRAWINGS

FIG. 1 is a graph showing cadmium concentrations in grains of 131 lines of rice cultivated in a field free of cadmium contamination.

FIG. 2 is a graph showing cadmium concentrations in the shoots and brown rice of Anjana Dhan and Nipponbare both cultivated in a field free of cadmium contamination.

FIG. 3 is a graph showing cadmium concentrations in the shoots and brown rice of Anjana Dhan and Nipponbare both cultivated in a cadmium-contaminated soil.

FIG. 4 is a graph showing relative levels of cadmium concentration in the shoots of an F₂ population, where the concentration of cadmium in the shoots of Anjana Dhan is 100. In FIG. 4, “A”, “H”, and “B” represent the genotypes of markers near which the respective quantitative trait loci were detected. “A”, “H”, and “B” indicate homozygous Anjana Dhan, heterozygous allele, and homozygous Nipponbare, respectively.

FIG. 5 shows results of PCR performed on seven rice cultivars that show high cadmium concentrations in the grains, namely Jarjan in the first lane, Anjana Dhan in the second lane, IR58 in the third lane, Bleiyo in the fourth lane, IR36 in the fifth lane, and PI312777 in the sixth lane.

FIG. 6 shows a result of a comparison between the amino acid sequence of OsHMA3n protein from Nipponbare and that of OsHMA3a protein from Anjana Dhan. In FIG. 6, TM1 to TM8 indicate transmembrane domains.

FIG. 7 is a graph showing results of quantitative PCR.

FIG. 8 shows results of functional analysis of OsHMA3 genes in yeast.

FIG. 9 is a graph showing results of measurement of cadmium concentrations in the xylem sap, shoots, and roots of Anjana Dhan and Nipponbare over time, (a), (b), and (c) showing the results of measurement in the xylem sap, in the shoots, and in the roots, respectively.

FIG. 10 is a graph showing cadmium absorbing power of Anjana Dhan and Nipponbare in the roots.

FIG. 11 is a graph showing results of measurement of cadmium concentrations in the xylem sap of Anjana Dhan and Nipponbare.

FIG. 12 shows results of a complementation test, (a) and (b) showing cadmium concentrations in the shoots and roots, respectively, of three transgenic lines carrying OsHMA3n in their Anjana Dhan backgrounds and a control line carrying an empty vector in its Anjana Dhan background.

FIG. 13 shows concentrations of micronutrients in the three transgenic lines carrying OsHMA3n genes in their Anjana Dhan backgrounds and the control line carrying an empty vector in its Anjana Dhan background.

FIG. 14 shows concentrations of macronutrients in the three transgenic lines carrying OsHMA3n genes in their Anjana Dhan backgrounds and the control line carrying an empty vector in its Anjana Dhan background.

FIG. 15 shows results of measurement of levels of cadmium accumulation in RNAi lines of Nipponbare in which OsHMA3 gene expression was knocked down by RNAi and a control line carrying an empty vector in its Nipponbare background, (a) and (b) showing cadmium concentrations in the shoots and roots, respectively, of the three RNAi lines and the control line.

FIG. 16 shows concentrations of micronutrients in the RNAi lines of Nipponbare in which OsHMA3 gene expression was knocked down by RNAi and the control line carrying an empty vector in its Nipponbare background, (a) showing levels of OsHMA3 gene expression in the three RNAi lines and the control line, (b) showing concentrations of micronutrients in the shoots and roots of the RNAi and control lines.

FIG. 17 shows an expression pattern of OsHMA3 genes, (a) showing the expression of two allelic genes of OsHMA3 in the roots and shoots of Nipponbare and Anjana Dhan, (b) showing the expression of two allelic genes in different parts of the roots of Nipponbare and Anjana Dhan.

FIG. 18 shows the localization of OsHMA3 protein in rice roots, (a) and (b) showing results of immunostaining in the roots of Anjana Dhan and Nipponbare, respectively.

FIG. 19 shows subcellular localization of OsHMA3n protein, (a) showing fluorescence of GFP in onion epidermal cells expressing GFP-OsHMA3n protein, (b) showing fluorescence of GFP in onion epidermal cells expressing GFP protein alone.

FIG. 20 shows results of Western blot analysis, (a) showing the specificity of an anti-OsHMA3n antibody, (b) showing results of sucrose-density gradient analysis.

FIG. 21 shows results of functional analysis in yeast for cadmium, (a) showing the growth of wild-type yeast cells (BY4741 strain) transformed with a pYES2 vector alone, OsHMA3n gene, OsHMA3a gene, AtHMA3 gene, and two chimeric genes, respectively, where these wild-type yeast cells were grown in the presence of glucose, (b) showing the growth of wild-type yeast cells (BY4741 strain) transformed with a pYES2 vector alone, OsHMA3n gene, OsHMA3a gene, AtHMA3 gene, and two chimeric genes, respectively, where these wild-type yeast cells were grown in the presence of galactose.

FIG. 22 schematically shows chimera proteins between OsHMA3n protein and OsHMA3a protein.

FIG. 23 shows results of functional analysis in yeast for cadmium, (a) showing the growth of Δycf1 yeast cells transformed with a pYES2 vector alone, OsHMA3n gene, OsHMA3a gene, and AtHMA3 gene, respectively, where these Δycf1 yeast cells were grown in the presence of glucose, (b) showing the growth of Δycf1 yeast cells transformed with a pYES2 vector alone, OsHMA3n gene, OsHMA3a gene, and AtHMA3 gene, respectively, where these Δycf1 yeast cells were grown in the presence of galactose.

FIG. 24 shows results of functional analysis in yeast for metals, (a) showing the growth of Δzrc1 yeast strains transformed with a pYES2 vector alone, OsHMA3n gene, OsHMA3a gene, and AtHMA3 gene as a positive control, respectively, where these Δzrc1 yeast strains were grown in the presence of galactose and 4 mM ZnSO₄, (b) showing the growth of Δcot1 yeast strains transformed with a pYES2 vector alone, OsHMA3n gene, OsHMA3a gene, and AtHMA3 gene as a positive control, respectively, where these Δcot1 yeast strains were grown in the presence of galactose and 2.5 mM CoCl₂, (c) showing the growth of wild-type yeast cells transformed with a pYES2 vector alone, OsHMA3a gene, OsHMA3aH80R gene, OsHMA3aV638A gene, OsHMA3aH80R/V638A gene, and OsHMA3n gene, respectively, where these wild-type yeast strains were grown in the presence of galactose and 20 μM CdSO₄.

FIG. 25 shows an effect of OsHMA3n gene overexpression on the accumulation of cadmium and on the accumulation of other metals, (a), (b), and (c) showing the concentrations of cadmium, zinc, and iron in the brown rice, respectively.

FIG. 26 shows levels of OsHMA3n gene expression in over-expression lines and in a vector control line.

FIG. 27 shows an effect of OsHMA3n gene overexpression on the accumulation of cadmium and on the accumulation of other metals, (a), (b), and (c) showing the concentrations of cadmium, zinc, and iron in the shoots, respectively.

### DESCRIPTION OF EMBODIMENTS

An embodiment of the present invention is described in detail below. It should be noted that the present invention is not to be limited to such an embodiment, but may be implemented in aspects with various modifications within the scope of description. Further, all of the academic literatures and patent literatures described in this specification are incorporated herein by references in this specification. It should be noted that in this specification, the range of numerical values “A to B” means “not less than A and not more than B”, unless otherwise noted.

[1. Method for Producing a Transgenic Plant]

A method according to the present invention for producing a transgenic plant is a method for producing a transgenic plant changed in localization of cadmium accumulation, comprising the step of introducing a polynucleotide into a plant such that the polynucleotide is able to be expressed, the polynucleotide being: (a) a polynucleotide encoding a polypeptide consisting of the amino acid sequence of SEQ ID NO: 1; (b) a polynucleotide encoding a polypeptide in which one or several amino acids are substituted, deleted, and/or added in the amino acid sequence of SEQ ID NO: 1 and which has activity to change the localization of cadmium accumulation; (c) a polynucleotide encoding a polypeptide consisting of the amino acid sequence of SEQ ID NO: 2; or (d) a polynucleotide encoding a polypeptide in which one or several amino acids are substituted, deleted, and/or added in the amino acid sequence of SEQ ID NO: 2 and which has activity to change the localization of cadmium accumulation.

The inventor of the present invention newly identified in rice genes (OsHMA3n gene and OsHMA3a gene) responsible for the transport of cadmium into cells. The polypeptide of SEQ ID NO: 1 and the polypeptide of SEQ ID. NO. 2 are translational products of OsHMA3n gene and OsHMA3a gene, respectively. That is, the polypeptide consisting of the amino acid sequence of SEQ ID NO: 1 and the polypeptide consisting of the amino acid sequence of SEQ ID NO: 2 are proteins newly identified in rice by the inventor of the present invention to be responsible for the transport of cadmium into cells. That is, the polypeptide consisting of the amino acid sequence of SEQ ID NO: 1 and the polypeptide consisting of the amino acid sequence of SEQ ID NO: 2 can be said to have the activity to change the localization of cadmium accumulation. In this specification, OsHMA3n gene and OsHMA3a gene are sometimes referred to simply as “OsHMA3 gene”, and OsHMA3n protein and OsHMA3a protein are sometimes referred to simply as “OsHMA3 protein”.

Examples of the polynucleotide (a) or (b) encompass a polynucleotide having the base sequence of SEQ ID NO: 3. Further, one possible example of the polynucleotide (c) or (d) is a polynucleotide having a base sequence of SEQ ID NO: 4.

It should be noted here that in this specification, the phrase “with a change (changed) in localization of cadmium accumulation” means that the transgenic plant has such a change in localization of cadmium accumulation such that cadmium accumulation is more localized in the roots or such that cadmium accumulation is more localized in those parts of the plant except for the roots as compared with a wild-type plant having none of the polynucleotides (a) to (d) introduced thereinto. Further, “those parts of the plant except for the roots” means those parts of the plant other than the roots, such as a stalk, leaves, and grains. It should be noted that in this specification, “those parts of the plant except for the roots” are sometimes referred to as “shoots”.

Such a change in localization of cadmium accumulation can be confirmed by growing the wild-type plant and the transgenic plant under the same conditions and comparing levels of cadmium accumulation in the roots or in those parts of the plants except for the roots between the wild-type and transgenic plants thus grown. The levels of cadmium accumulation can be measured, for example, by a method to be described later in Examples.

The “cadmium” may be ionized cadmium or cadmium forming a salt. Further, the “cadmium” refers both to cadmium and a compound containing cadmium.

Further, the “polypeptide” can be reworded as “peptide” or “protein”. The clause “one or several amino acids are substituted, deleted, and/or added” means a substitution, deletion, and/or addition of as many amino acids (e.g., 20 amino acids or less, preferably 10 amino acids or less, more preferably 7 amino acids or less, still more preferably 5 amino acids or less, or especially preferably 3 amino acids or less) as can be substituted, deleted, or added by a publicly known mutant polypeptide generation method such as site-directed mutagenesis. Such a mutant polypeptide is not to be limited to a polypeptide having a mutation artificially introduced by a publicly known mutant polypeptide generation method, but may be a polypeptide obtained by isolating and purifying a naturally-occurring similar mutant polypeptide.

Further, the “polynucleotide”, which can be reworded as “nucleic acid” or “nucleic acid molecule”, means a polymer of nucleotides. Further, the “base sequence”, which can be reworded as “nucleic acid sequence” or “nucleotide sequence”, is represented as a sequence of deoxyribonucleotides (which are abbreviated as A, G, C, and T). Further, the “polynucleotide consisting of a base sequence of SEQ ID NO: 1” indicates a polynucleotide consisting of a sequence of deoxynucleotides A, G, C, and/or T of SEQ ID NO: 1.

There is no particular limit to how the polynucleotide is obtained. For instance, the polynucleotide can be obtained by a publicly known technique. For example, the polynucleotide can be obtained by a method using amplification means such as PCR. Specifically, a large amount of a DNA fragment containing the polynucleotide can be obtained by generating primers respectively corresponding to sequences (or sequences complementary thereto) at the 5′ and 3′ ends of any one of the polynucleotides (a) to (d), carrying out PCR or the like by use of these primers with genomic DNA (or cDNA) as a template, and thereby amplifying a DNA region between the primers.

Alternatively, the polynucleotide can also be obtained by designing, on the basis of publicly known sequence information on Nipponbare, a primer that allows an OsHMA3 gene region to be amplified, and amplifying the OsHMA3 gene region by use of the primer with genomic DNA (or cDNA) or an RT-PCR product as a template.

Alternatively, the polynucleotide can also be obtained by isolating and cloning a DNA fragment containing either the polynucleotide or an oligonucleotide containing part of the sequence of the polynucleotide For example, the polynucleotide can also be obtained by preparing a probe that hybridizes specifically with part of the base sequence of any one of the polynucleotides (a) to (d) and screening a genomic DNA library or a cDNA library with the probe. Such a probe may be of any sequence and/or length, provided the probe hybridizes specifically with at least part of either the base sequence of the polynucleotide or a sequence complementary thereto.

Although there is no particular limit to a supply source from which the polynucleotide is obtained, it is preferable that such a supply source be a poaceous plant (rice, maize, etc.). This is because the polypeptide (OsHMA3 protein) consisting of the amino acid sequence of SEQ ID NO: 1 and the polypeptide (OsHMA3 protein) consisting of the amino acid sequence of SEQ ID NO: 2 are derived from Nipponbare, which is a rice cultivar.

The “polynucleotide” may be introduced into the plant in any form, provided that the “polynucleotide” can be expressibly introduced into the plant. For example, the “polynucleotide” may be introduced in the form of RNA (e.g., mRNA) or in the form of DNA (e.g., cDNA or genomic DNA).

The “plant” into which the polynucleotide is introduced is not to be particularly limited, but can be appropriately selected for any purpose. Possible examples of the “plant” encompass poaceous plants, solanaceous plants, papilionaceous plants, etc. Possible examples of the “poaceous plants” encompass rice, barley, wheat, maize, rye, sorghum, etc. Possible examples of the “solanaceous plants” encompass eggplant, etc. Possible examples of the “papilionaceous plants” encompass soybean, etc.

The method according to the present invention for producing a transgenic plant changed in localization of cadmium accumulation is not to be particularly limited, provided the method causes expression of either any one of the polynucleotides (a) to (d) or a polypeptide carrying a recombinant expression vector containing the polynucleotide and having activity to change the localization of cadmium accumulation. It should be noted that in this specification, the “activity to change the localization of cadmium accumulation” means “activity to change the localization of cadmium accumulation such that cadmium accumulation is more localized in the roots or such that cadmium accumulation is more localized in those parts of the plant except for the roots as compared with a wild-type plant having none of the polynucleotides (a) to (d) introduced thereinto”.

The “recombinant expression vector” is not particularly limited in type, provided that it contains any one of the polynucleotides (a) to (d). A possible example of the “recombinant expression vector” is a recombinant expression vector with an insertion of cDNA of SEQ ID NO: 3 or 4. The “recombinant expression vector” can be prepared from a plasmid, a cosmid, or the like. However, the present invention is not limited to these.

Further, the “recombinant expression vector” is not to be particularly limited, provided it allows the inserted gene to be expressed in a cell of the plant (such a cell being hereinafter referred to as “host cell”). For example, in a case where the recombinant expression vector is introduced into the plant by a method using Agrobacterium (Agrobacterium infection method), it is preferable that the recombinant expression vector be a pBI binary vector or the like. Possible examples of such a binary vector encompass pBIG, pBIN19, pBI101, pBI121, pBI221, etc.

Further, it is preferable that the “recombinant expression vector” be a vector having a promoter that allows a gene to be expressed in a cell of the plant (target plant) into which the “recombinant expression vector” is introduced. As the promoter, a publicly known promoter can be suitably used, possible examples of which encompass a cauliflower mosaic virus 35S promoter (CaMV35S), a ubiquitin promoter, an actin promoter, etc. By using a recombinant expression vector obtained by incorporating such a promoter sequence and any one of the polynucleotides (a) to (d) into a plasmid or the like, the introduced polynucleotide can be suitably expressed in a cell of the plant. Among these promoters, it is preferable that the cauliflower mosaic virus 35S promoter be used, because the cauliflower mosaic virus 35S promoter helps achieve a high level of expression of OsHMA3 protein. For example, achieving a high level of expression of OsHMA3n protein allows production of a transgenic plant in shoots of which cadmium is less easily accumulated. Further, achieving a high level of expression of OsHMA3a protein allows production of a transgenic plant in shoots of which cadmium is more easily accumulated.

Further, the inventor of the present invention found that OsHMA3 protein is expressed mainly in plant roots. Accordingly, for the purpose of allowing OsHMA3 protein to suitably function in the resulting transgenic plant, it is preferable to introduce any one of the polynucleotides (a) to (d) into the plant such that the polynucleotide is expressible specifically in the roots of the plant.

For example, by introducing any one of the polynucleotides (a) to (d) into the plant under the control of a promoter known to regulate a gene that is expressed specifically in the roots, it can be made possible to allow the polynucleotide to be expressed specifically in the roots of the plant. As such a promoter, an OsHMA3 promoter can be used, for example.

There is no particular limit to a method for introducing the polynucleotide or the recombinant expression vector into the plant, i.e., to a method of transformation. For example, the polynucleotide or the recombinant expression vector may be incorporated into a chromosome, or the polynucleotide may be incorporated into a specific site of a chromosome by homologous recombination. Alternatively, the polynucleotide or the recombinant expression vector may be transiently expressed in the plant.

As the method of transformation, a conventionally publicly known genetic engineering method (genetic manipulation technique) may be used, suitable examples of which encompass an Agrobacterium infection method, an electroporation method, a calcium-phosphate method, a protoplast method, a lithium-aceate method, a particle gun method, etc. For example, as the “Agrobacterium infection method”, a method described in Plant, J. 6: 271-282 (1994) can be used.

Further, whether or not the polynucleotide or the recombinant expression vector has been introduced into a host cell and, further, whether or not the polynucleotide or the recombinant expression vector is surely expressed in the host cell can be confirmed by using any of various markers. An example is a method for, with use as a marker of a drug resistance gene that imparts resistance to an antibiotic substance such as Hygromycin, introducing into a host cell an expression vector obtained by incorporating the marker and any one of the polynucleotides (a) to (d) into a plasmid or the like. Use of this method makes it possible to confirm, by selection of drug, whether or not the introduced gene is surely expressed in the host cell.

A transgenic plant produced by the method according to present invention for producing a transgenic plant is a plant having any one of the polynucleotides (a) to (d) or the recombinant expression vector introduced thereinto and expressing a polypeptide having activity to change the localization of cadmium accumulation. That is, the transgenic plant produced by the method according to present invention for producing a transgenic plant is different in localization of cadmium accumulation from a wild-type plant having none of the polynucleotides (OsHMA3 genes) (a) to (d) introduced thereinto.

More specifically, the polynucleotide (a) or (b) corresponds to OsHMA3n gene derived from Nipponbare. For this reason, it is considered that a transgenic plant having the polynucleotide (a) or (b) introduced thereinto more accumulates cadmium in the roots and less accumulates cadmium in those parts (e.g., grains, etc.) of the plant except for the roots than does a wild-type plant having neither of these polynucleotides introduced thereinto.

Meanwhile, the polynucleotide (c) or (d) corresponds to OsHMA3a gene derived from Anjana Dhan. For this reason, it is considered that a transgenic plant having the polynucleotide (c) or (d) introduced thereinto less accumulates cadmium in the roots and more accumulates cadmium in those parts (e.g., grains, etc.) of the plant except for the roots than does a wild-type plant having neither of these polynucleotides introduced thereinto.

Therefore, it can be said that a transgenic plant having any one of the polynucleotides (a) to (d) introduced therein is different in localization of cadmium accumulation from a wild-type plant having none of these polynucleotides introduced thereinto.

The method according to the present invention for producing a transgenic plant makes it possible to easily produce a transgenic plant changed in localization of cadmium accumulation.

In the method according to the present invention for producing a transgenic plant, it is preferable that the plant be a poaceous plant.

[2. Transgenic Plant]

A transgenic plant according to the present invention is produced by the method according to the present invention for producing a transgenic plant.

The “method according to the present invention for producing a transgenic plant” has been described above in section “1. Method for Producing a Transgenic Plant”, and as such, is not described here.

The scope of the transgenic plant according to the present invention encompass not only a plant but also various forms of plant cells, e.g., suspended cultured cells, protoplasts, segments of leaves, calluses, etc. Further, once a transgenic plant having any one of the polynucleotides (a) to (d) incorporated into a chromosome of the plant is obtained by the method according to the present invention for producing a transgenic plant, the plant produces seeds having the polynucleotide introduced thereinto. Therefore, the present invention encompasses the seeds obtained from the transgenic plant.

Since the transgenic plant according to the present invention has a change in localization of cadmium accumulation, it can be used for various purposes. For example, a transgenic plant with such a change in localization of cadmium accumulation that cadmium accumulation is more localized in the roots makes it difficult for cadmium to be accumulated in those parts of the plant except for the roots. With this feature, the transgenic plant with such a change in localization of cadmium accumulation can be cultivated even in a cadmium-containing soil.

Furthermore, cadmium contained in the soil can be efficiently accumulated in the transgenic plant when the transgenic plant is cultivated in a cadmium-containing soil, the transgenic plant having been changed in localization of cadmium accumulation such that cadmium accumulation is more localized in those parts of the transgenic plant except for the roots. For this reason, the transgenic plant with this feature can be used to remove cadmium from a cadmium-containing soil. For example, cadmium can be efficiently removed from a cadmium-containing soil by repeating the step of growing, in the cadmium-containing soil, a transgenic plant with such a change in localization of cadmium accumulation that cadmium accumulation is more localized in those parts of the transgenic plant except for the roots, the step of harvesting the transgenic plant thus grown, and the step of disposing of the transgenic plant by incineration or the like.

[3. Kit]

A kit according to the present invention is a kit for producing a transgenic plant changed in localization of cadmium accumulation, comprising a polynucleotide, the polynucleotide being: (a) a polynucleotide encoding a polypeptide consisting of the amino acid sequence of SEQ ID NO: 1; (b) a polynucleotide encoding a polypeptide in which one or several amino acids are substituted, deleted, and/or added in the amino acid sequence of SEQ ID NO: 1 and which has activity to change the localization of cadmium accumulation; (c) a polynucleotide encoding a polypeptide consisting of the amino acid sequence of SEQ ID NO: 2; or (d) a polynucleotide encoding a polypeptide in which one or several amino acids are substituted, deleted, and/or added in the amino acid sequence of SEQ ID NO: 2 and which has activity to change the localization of cadmium accumulation.

The “polynucleotide” has been described above in section “1. Method for Producing a Transgenic Plant”, and as such, is not described here.

The kit according to the present invention may include a component other than the polynucleotide. For example, the kit according to the present invention may include a plasmid from which a recombinant expression vector containing the polynucleotide is prepared, a reagent necessary for producing the recombinant expression vector, a buffer, a reagent necessary for transforming the plant, etc.

Use of the kit according to the present invention makes it possible to easily produce a transgenic plant changed in localization of cadmium accumulation.

The present invention is not limited to the description of the embodiments above, but may be altered by a skilled person within the scope of the claims. An embodiment based on a proper combination of technical means disclosed in different embodiments is encompassed in the technical scope of the present invention.

### EXAMPLES

In the following, the present invention is described in more detail by way of Examples. However, the present invention is not to be limited to these Examples.

(Method for Measuring Cadmium Concentrations)

In the Examples of the present invention, cadmium concentrations in roots, shoots, and grains (brown rice) of rice were measured by chemically decomposing each plant sample with a concentrated nitric acid solution (60%) and measuring the absorbance of the decomposed plant sample with an atomic absorption photometer.

(1. Identification of a Gene Responsible for Shoot Cadmium Accumulation)

First, with various rice cultivars from all over the world (a total of 131 lines of rice), the level of cadmium accumulation in grains of each of the rice lines was determined. Specifically, cadmium concentrations in grains of rice cultivated in a filed free of cadmium contamination (with a cadmium concentration of less than 1 ppm in the soil) were measured.

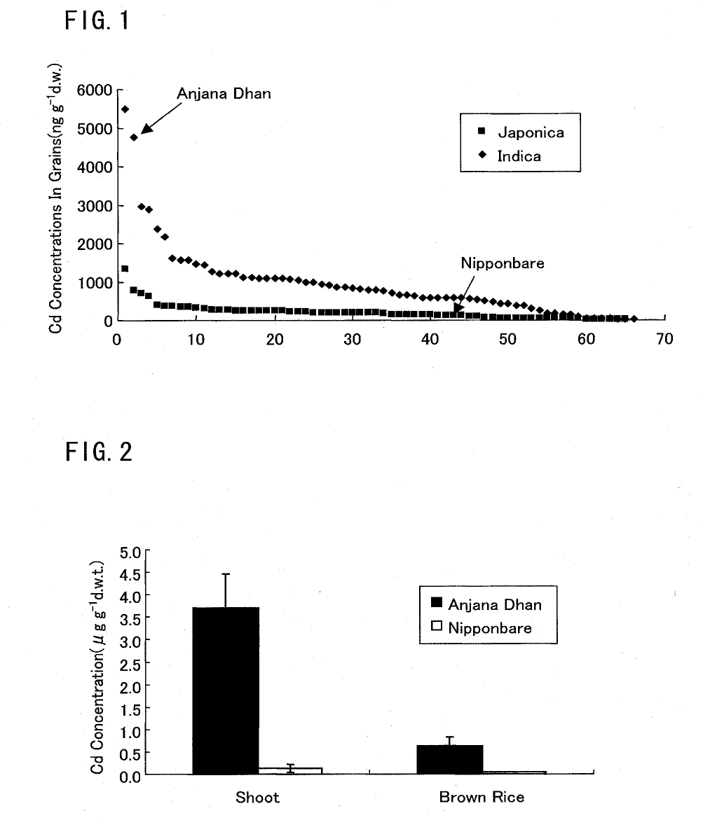

FIG. 1 is a graph showing cadmium concentrations in grains of 131 lines of rice cultivated in a field free of cadmium contamination. FIG. 1 clearly shows that there is a large variation in grain cadmium concentration among the rice lines. Above all, FIG. 1 clearly shows that Anjana Dhan, which is one line of indica, is 40 times or more as high in grain cadmium concentration as Nipponbare, which is one line of japonica.

Accordingly, with Anjana Dhan selected as a high cadmium-accumulating line and Nipponbare as a low cadmium-accumulating line, the levels of cadmium accumulation in Anjana Dhan and Nipponbare were determined in more detail. FIG. 2 is a graph showing cadmium concentrations in the shoots and brown rice of Anjana Dhan and Nipponbare both cultivated in a field free of cadmium contamination. It should be noted that the “shoots” refer to those parts of rice which are exposed above the ground, i.e., which include stalks and leaves, and do not include grains. FIG. 2 clearly shows that Anjana Dhan is 28 times as high in cadmium concentration as Nipponbare in the shoots, and 15 times as high in the brown rice.

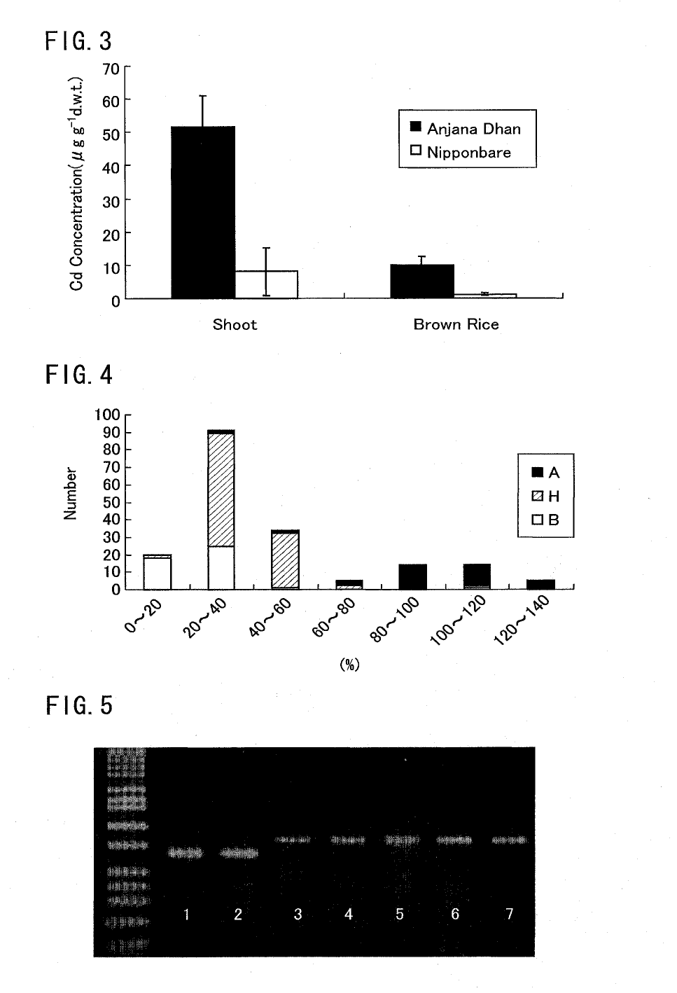

Next, the levels of cadmium accumulation in Anjana Dhan and Nipponbare both cultivated for 179 days with use of 1/5000a wagner pots in an artificially cadmium-contaminated soil were determined. The artificially cadmium-contaminated soil used had a cadmium concentration of 7.6 ppm in a fraction extracted with 0.1 N hydrochloric acid. FIG. 3 is a graph showing cadmium concentrations in the shoots and brown rice of Anjana Dhan and Nipponbare both cultivated in a cadmium-contaminated soil. FIG. 3 clearly shows that Anjana Dhan is 6 times as high in cadmium concentration than Nipponbare in the shoots, and 8 times as high in the brown rice.

Next, quantitative traits responsible for cadmium accumulation were identified by using an F₂ population derived from a cross between Anjana Dhan and Nipponbare. Specifically, the F₂ population, which had been obtained by using a ½ Kimura B solution containing 50 nM of cadmium, was grown for 10 days, and the cadmium concentrations in the shoots were measured. It should be noted that the ½ Kimura B solution is a culture medium containing macronutrients (mM), namely MgSO₄(0.28), (NH₄)₂SO₄(0.18), Ca(NO₃)₂(0.18), KNO₃(0.09), and KH₂PO₄(0.09), and micronutrients (mM), namely Fe(II)SO₄(10), H₃BO₃(3), MnCl₂(0.5), CuSO₄(0.2), ZnSO₄(0.4), and (NH₄)₆Mo₇O₂₄(1). The ½ Kimura B solution had its pH adjusted to 5.4 with 1N NaOH.

FIG. 4 is a graph showing relative levels of cadmium concentration in the shoots of the F₂ population, where the concentration of cadmium in the shoots of Anjana Dhan is 100. In FIG. 4, “A”, “H”, and “B” represent the genotypes of markers near which the respective quantitative trait loci (QTLs) were detected. “A”, “H”, and “B” indicate homozygous Anjana Dhan, heterozygous allele, and homozygous Nipponbare, respectively. As shown in FIG. 4, the shoot cadmium concentration divided the F₂ population in a ratio of 1:3. This result clearly shows that the difference in shoot cadmium concentration between Anjana Dhan and Nipponbare is attributed to a single genetic difference.

Accordingly, QTL analysis was carried out to detect quantitative trait loci controlling levels of cadmium accumulation in the shoots of the F₂ population. The QTL analysis was carried out by using qtl cartographer version 2.5 (provided from the Bioinformatics Research Center of North Carolina State University, http://statgen.ncsu.edu/qtlcart/WQTLCart.htm). In the result, it became clear that a QTL responsible for shoot cadmium accumulation resides on. This QTL had an LOD value of 53.1 and a contributing rate of 72.5%.

Furthermore, mapping of the causative gene was carried out by using 1000 F₂ plants or more, whereby a candidate region in which the QTL responsible for shoot cadmium accumulation resides was specified to be located within a region of 500 kb between markers RM21251 and RM21275. RAP-DB (http://rapdb.dna.affrc.go.jp/), which is a database that includes and discloses annotated information on rice genome, was searched for genes present in that region. In the result, it became clear that there are candidate genes in the region between the markers RM21251 and RM21275. Among them, OsHMA3 was deduced to be a gene responsible for cadmium transport, because the base sequences of Anjana Dhan and Nipponbare are different from each other as to OsHMA3.

Next, cDNAs were generated from six lines that show high cadmium concentration in the grains and from Nipponbare, all of which lines are listed in Table 1, and PCR was carried out with the cDNAs as templates, respectively.

TABLE 1

| | | Cadmium Concentrations |
|---|---|---|
| Lane No. | Names of Cultivars | in Grains (ng/g) |
| 1 | Jarjan | 5502 |
| 2 | Anjana Dhan | 4751 |
| 3 | IR58 | 2973 |
| 4 | Bleiyo | 2880 |
| 5 | IR36 | 2368 |
| 6 | PI312777 | 2174 |
| 7 | Nipponbare | 115 |

Specifically, the PCR was carried out with a primer designed to include a region of Nipponbare from a C terminus thereof to OsHMA3, i.e., a region of Nipponbare with which region a PCR amplification product of 848 bp would be produced. A reaction liquid for the PCR was prepared from Ex Taq (product name; manufactured by TaKaRa), and amplification was carried out by using Mastercycler (product name; manufactured by Eppendorf). Reaction conditions for the PCR were as follows: initial denaturation, 94° C., 20 seconds; denaturation, 98° C., 10 seconds; annealing, 62° C., 30 seconds; and elongation, 72° C., 60 seconds. The process from denaturation to elongation was repeated 35 times.

FIG. 5 shows results of the PCR performed on the seven rice cultivars that show high cadmium concentrations in the grains, namely Jarjan in the first lane, Anjana Dhan in the second lane, IR58 in the third lane, Bleiyo in the fourth lane, IR36 in the fifth lane, and PI312777 in the sixth lane. FIG. 5 clearly shows that Jarjan and Anjana Dhan, which are top two cultivars that showed high cadmium concentrations in the grains, are short in length of their respective PCR amplification products.

(2. Isolation of OsHMA3 Genes)

OsHMA3 genes were isolated from Anjana Dhan and Nipponbare, respectively, in the following manner. Specifically, an ORF of OsHMA3n gene derived from Nipponbare was isolated by designing primers for the N and C termini on the basis of information from the database (RAP-DB) and amplifying the gene by PCR. An ORF of OsHMA3a gene derived from Anjana Dhan was isolated by carrying out 5′-RACE and 3′-RACE with a SMART RACE cDNA Amplification kit (product name; manufactured by Clonetech).

The amino acid sequence of OsHMA3 protein derived from Nipponbare (OsHMA3n protein) is shown in SEQ ID NO: 1, and the base sequence of OsHMA3 gene derived from Nipponbare (OsHMA3n gene) is shown in SEQ ID NO: 3. Further, the amino acid sequence of OsHMA3 protein derived from Anjana Dhan (OsHMA3a protein) is shown in SEQ ID NO: 2, and the base sequence of OsHMA3 gene derived from Anjana Dhan (OsHMA3a gene) is shown in SEQ ID NO: 4.

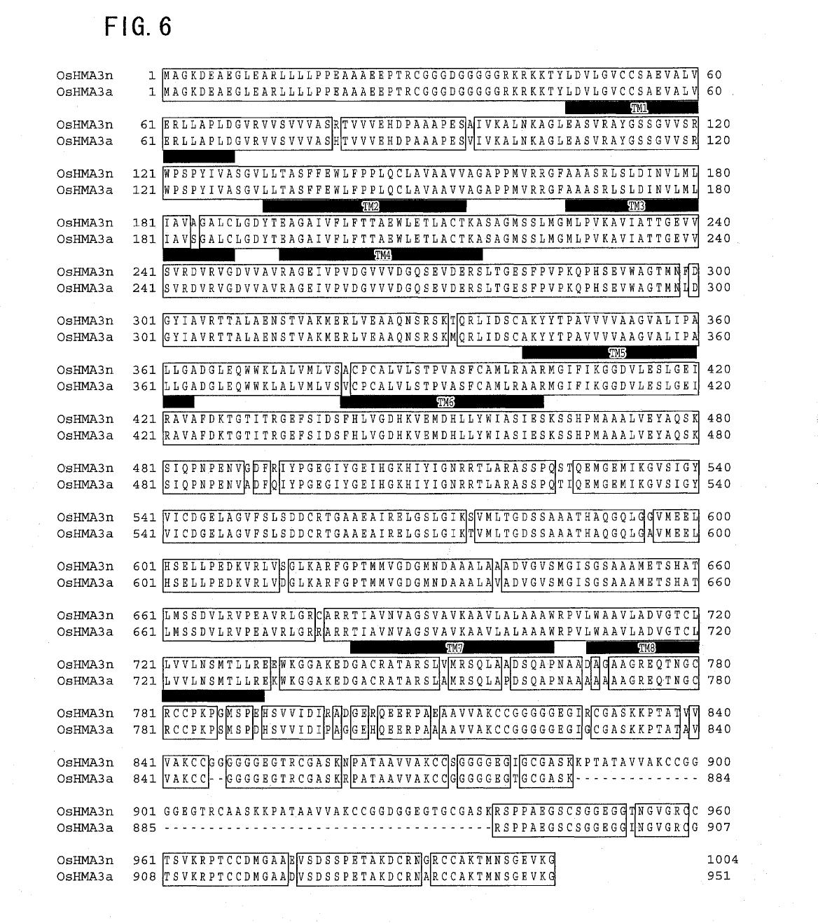

FIG. 6 shows a result of a comparison between the amino acid sequence of OsHMA3n protein from Nipponbare and that of OsHMA3a protein from Anjana Dhan. In FIG. 6, TM1 to TM8 indicate transmembrane domains. As shown in FIG. 6, OsHMA3a protein derived from Anjana Dhan has a 53-amino acid deletion in the C-terminal region as compared with OsHMA3n protein derived from Nipponbare. This suggested a possibility that OsHMA3a protein derived from Anjana Dhan does not function properly. It should be noted that OsHMA3a protein and OsHMA3n protein had a homology of 91.4% with each other.

(3. Functional Analysis of OsHMA3 Genes)

First, the effect of cadmium addition on OsHMA3 gene expression was analyzed. Specifically, seedlings 11 days after seeding were cultured using a ½ Kimura B solution containing 50 nM of cadmium for 24 hours. As a negative control, seedlings 11 days after seeding were cultured using a ½ Kimura B solution free of cadmium. After the culturing, RNAs were extracted from shoots and roots. From the RNAs thus obtained, cDNAs were synthesized by reverse transcriptional reaction, respectively. Quantitative PCR was carried out with the synthesized cDNAs as templates, respectively.

It should be noted that the total RNAs were extracted by using a Qiagen's RNeasy Plant Mini Kit (product name; manufactured by Qiagen) and the cDNAs were synthesized by using an Invitrogen's SuperScript II reverse transcriptase (product name; manufactured by Invitrogen). A PCR reaction liquid for the quantitative PCR was prepared from Thunderbrid SYBR qPCR Mix (product name; manufactured by Toyobo), and amplification was carried out by using Mastercycler ep Realprex real time PCR (product name; manufactured by Eppendorf). Reaction conditions for the PCR were as follows: initial denaturation, 95° C., 30 seconds; denaturation, 95° C., 30 seconds; annealing, 62° C., 20 seconds; and elongation, 72° C., 35 seconds. The process from denaturation to elongation was repeated 40 times.

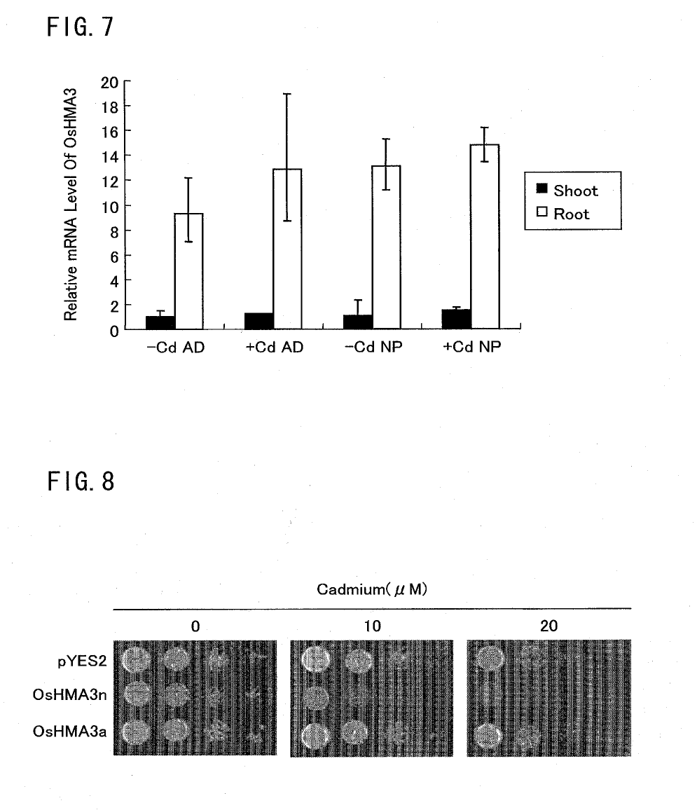

FIG. 7 is a graph showing results of the quantitative PCR. FIG. 7 clearly shows that OsHMA3 gene is expressed mainly in the roots. The addition of cadmium did not affect the levels of OsHMA3 gene expression. Furthermore, there was no significant difference in level of OsHMA3 gene expression between Anjana Dhan and Nipponbare regardless of whether cadmium was added. This result clearly shows that the difference in shoot cadmium concentration between Anjana Dhan and Nipponbare is not attributed to a difference in level of OsHMA3 gene expression but to a difference in function between the respective OsHMA3 proteins.

Next, OsHMA3 genes respectively isolated from Anjana Dhan and Nipponbare were expressed in yeast, whereby the transport activity of both OsHMA3 proteins for cadmium was analyzed. Specifically, OsHMA3 respectively derived from Anjana Dhan and Nipponbare were each introduced with an expression vector pYES2 (manufactured by Invitrogen) into yeast (BY4741 strain, purchased from Euroscarf) by a publicly known lithium-acetate method. A negative control was prepared by introducing an expression vector pYES2 alone into yeast.

The transformed yeast strains thus obtained were put in agar media respectively containing 0 μM, 10 μM, and 20 μM of cadmium and cultured at 30° C. for 42 hours. Specifically, first, the transformed yeast strains were cultured in liquid SC media containing 2% glucose and free of uracil until a logarithmic growth phase (OD 0.5 to 1.0) was reached. On the basis of the OD thus measured, the density of each of the strains was uniformized. The transformed yeast strains were each washed three times with sterile water, and then cultures of the transformed yeast strains were diluted with five-leveled concentrations serially varying by tenfold. Five microliters of each culture of the transformed yeast strains were spotted on each of the agar media. The agar media used were SC media free of uracil. For induction of OsHMA3 gene expression from a GAL1 promoter, galactose was used as a carbon source.

OsHMA3 protein acts as a discharging transporter, and in yeast expressing OsHMA3 protein, cadmium in the cytoplasm concentrates on a particular organelle. Therefore, it is assumed that the growth of yeast is inhibited by cadmium toxicity. On this assumption, the presence or absence of cadmium transport activity was determined by using the growth of yeast as an index.

FIG. 8 shows results of functional analysis of OsHMA3 proteins. As shown in FIG. 8, the yeast expressing OsHMA3n protein derived from Nipponbare showed inhibited growth in the cadmium-containing media as compared with the yeast carrying a pYES2 vector as a negative control. This result clearly shows that OsHMA3n protein derived from Nipponbare has cadmium transport activity. Meanwhile, the yeast expressing OsHMA3a protein derived from Anjana Dhan showed similar growth to the yeast carrying a pYES2 vector as a negative control. This result clearly shows that OsHMA3a protein derived from Anjana Dhan does not have cadmium transport activity.

Furthermore, OsHMA3n protein was physiologically analyzed. Specifically, Anjana Dhan and Nipponbare seedlings 28 days after seeding were grown using a ½ Kimura B solution containing 1 μM of cadmium. The xylem sap, the shoots, and the roots were sampled at various timings over a period of time, and the concentrations of cadmium in them were measured. The xylem sap were collected by cutting a sheath with a razor horizontally by 3 to 4 cm above a boundary line between the shoot and the roots and collecting, with a micropipette, sap exuding from the cut surface. First 1 to 2 microliters of each sap after the cutting were not collected, because such a portion of the sap might have a cytoplasmic fluid mixed with it.

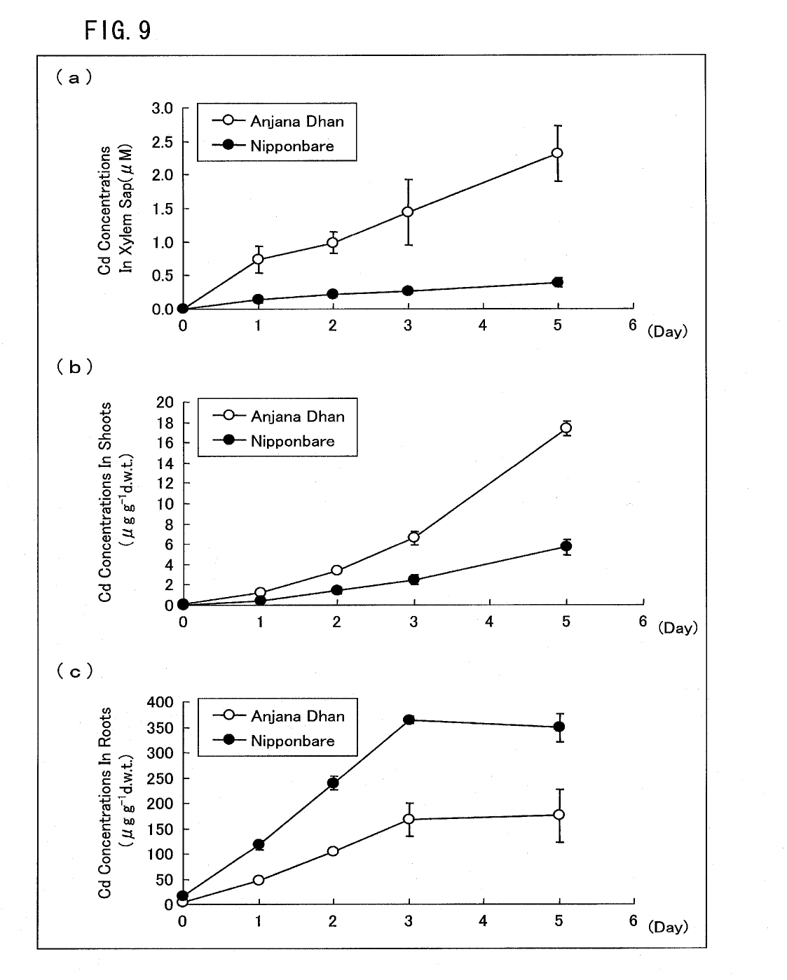

FIG. 9 is a graph showing results of measurement of cadmium concentrations in the xylem sap, shoots, and roots of Anjana Dhan and Nipponbare over time. In FIG. 9, (a), (b), and (c) show the results of measurement in the xylem sap, in the shoots, and in the roots, respectively. FIG. 9 clearly shows that on the fifth day of culturing, Anjana Dhan was 6 times as high in cadmium concentration as Nipponbare in the xylem sap, and 3 times as high in the shoots.

Meanwhile, it became clear that Nipponbare is twice as high in cadmium concentration as Anjana Dhan in the roots. These results indicate that Anjana Dhan has a lower ability to hold absorbed cadmium in the roots than Nipponbare.

Accordingly, absorbing power of Anjana Dhan and Nipponbare in the roots was analyzed. Specifically, the roots of Anjana Dhan and Nipponbare seedlings 21 days after seeding were exposed to ½ Kimura B solutions containing 0 μM to 5 μM of cadmium. After 30 minutes of exposure, the concentrations of cadmium absorbed in the roots were measured.

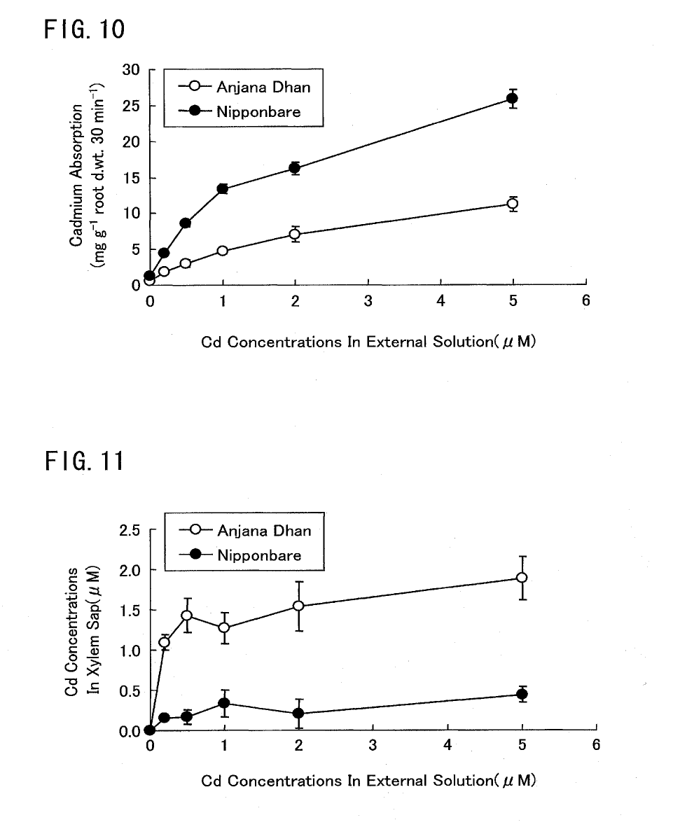

FIG. 10 is a graph showing cadmium absorbing power of Anjana Dhan and Nipponbare in the roots. Regardless of the cadmium concentrations of the solutions to which the roots of Anjana Dhan and Nipponbare were exposed, Nipponbare, which is a low cadmium-accumulating line, was higher in absorbing power than Anjana Dhan, which is a high cadmium-accumulating line. These results indicate that the high accumulation of cadmium in the shoots and grains of Anjana Dhan is not attributed to a high ability of absorbing cadmium from outside.

Accordingly, the concentrations of cadmium in the xylem sap of Nipponbare and Anjana Dhan was determined. Specifically, the roots of Anjana Dhan and Nipponbare seedlings 33 days after seeding were cultured for 3 days in ½ Kimura B solutions containing 0 μM to 5 μM of cadmium. After the culturing, the cadmium concentrations in the xylem sap were measured.

FIG. 11 is a graph showing results of measurement of cadmium concentrations in the xylem sap of Anjana Dhan and Nipponbare. Both cultivars, Nipponbare and Anjana Dhan, showed saturated concentrations of cadmium in the xylem sap when cultured in the culture solution containing 0.5 μM of cadmium. However, Anjana Dhan was 8.5 times as high in cadmium concentration as Nipponbare in the xylem sap.

These results of the physiological analysis indicate that the high accumulation of cadmium in the xylem sap of Anjana Dhan is due to that fact that Anjana Dhan became unable to hold cadmium in the roots and hence showed a higher level of cadmium discharge into the xylem. That is, it was considered that since Anjana Dhan is higher than Nipponbare both in activity to absorb cadmium through the roots and in activity to transport cadmium from the roots to the shoots, Anjana Dhan shows high accumulation of cadmium in the shoots and grains.

(4. Functional Analysis of Transgenic Lines)

The accumulation of cadmium in transgenic lines carrying OsHMA3n genes in their Anjana Dhan backgrounds was examined.

With Nipponbare genomic DNA as a template, a 6.8-kb DNA fragment having a 2.1-kb promoter and a full-length OsHMA3n gene was amplified by PCR. The two DNA fragments were amplified by using the following pairs of primers, respectively:

(SEQ ID NO: 5)
5′-atctagaAGCATAAAAGAATAGAGCCGTGGAC-3′
and
(SEQ ID NO: 6)
5′-GGATGCGTCAATCAGTTTACCA-3′;
and
(SEQ ID NO: 7)
5′-GGCACAATGAACTTTGACGGT-3′
and
(SEQ ID NO: 8)
5′-CTCTTCTGGACAAGCTTCCTTAATC-3′.

First, the two DNA fragments were cloned into a pTA2 vector, and then linked to each other via a restriction enzyme site Af1II. The resulting 6.8-kb DNA fusion was then inserted into pPZP2H-lac, which is a binary vector.

The resulting constructs thus generated were introduced into Anjana Dhan-derived calluses for transformation by a method using Agrobacterium tumefaciens (EHA101 strain).

After the introduction of plasmids, the transgenic plants were pre-cultured on gel for about 100 days. The transgenic seedlings were cultured in a nutrient solution for 1 to 3 weeks and then subjected to cadmium treatment. The cadmium treatment was performed by exposing the seedlings a nutrient solution containing 50 nM CdSO₄ for 10 days. The nutrient solution was changed once every two days.

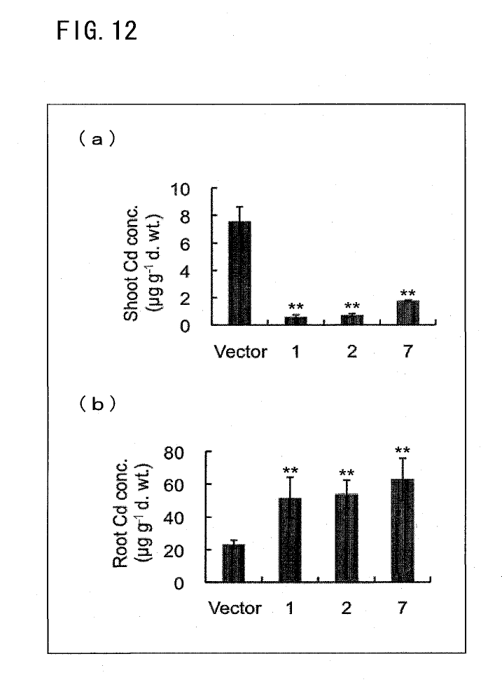

FIG. 12 shows results of a complementation test, (a) and (b) showing cadmium concentrations in the shoots and roots, respectively, of three transgenic lines carrying OsHMA3n in their Anjana Dhan backgrounds and a control line carrying an empty vector in its Anjana Dhan background. Data for (a) and (b) are mean±SD (n=3). A statistical analysis was carried out according to Dunnett's t-test. In the graph, the asterisk “*” means that there is a significant difference between the control and transgenic lines at a significance level of less than 5%, and the double asterisk “**” means that there is a significant difference between the control and transgenic lines at a significance level of less than 1%.

As shown in (a) of FIG. 12, the transgenic lines carrying OsHMA3n genes in their Anjana Dhan backgrounds showed significantly decreased cadmium accumulation in the shoots (p<0.01). On the other hand, as shown in (b) of FIG. 12, the transgenic lines carrying OsHMA3n genes in their Anjana Dhan backgrounds showed significantly increased cadmium accumulation in the roots (p<0.01).

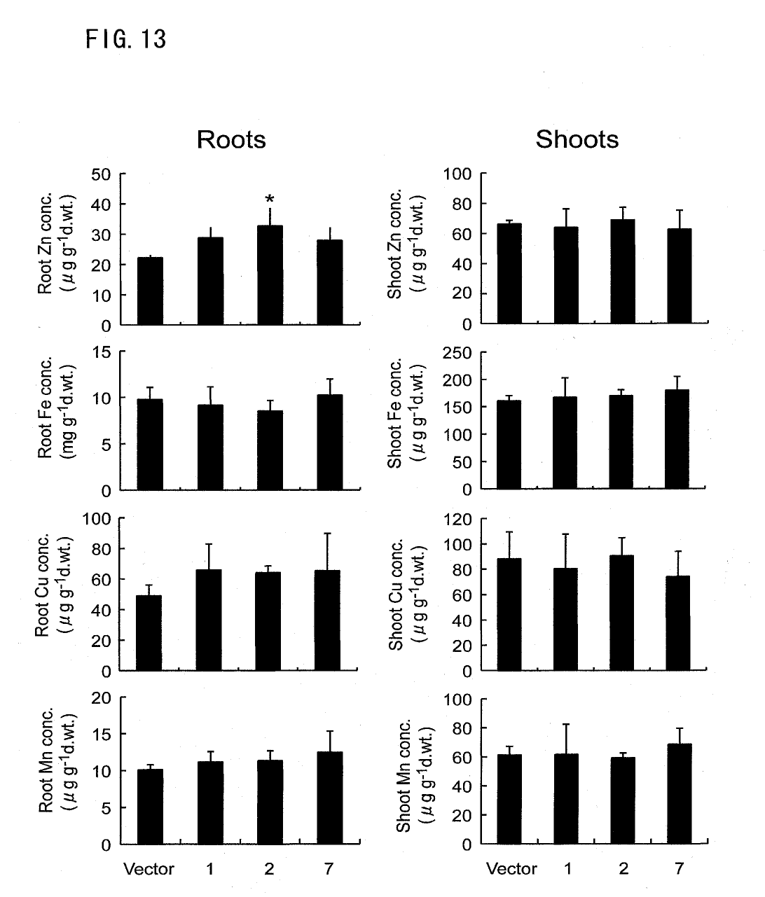

FIG. 13 shows concentrations of micronutrients in the three transgenic lines carrying OsHMA3n genes in their Anjana Dhan backgrounds and the control line carrying an empty vector in its Anjana Dhan background. The transgenic and control lines were exposed to 50 nM cadmium for 10 days. Data for the roots and shoots are mean±SD (n=4). A statistical analysis was carried out according to Dunnett's t-test. In the graph, the asterisk “*” means that there is a significant difference between the control and transgenic lines at a significance level of less than 5%.

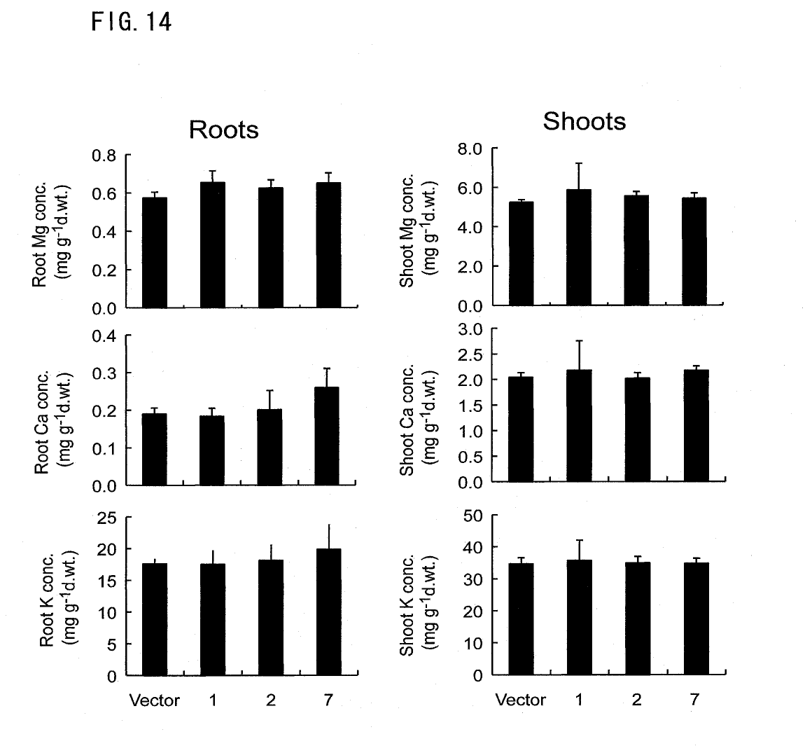

FIG. 14 shows concentrations of macronutrients in the three transgenic lines carrying OsHMA3n in their Anjana Dhan backgrounds and the control line carrying an empty vector in its Anjana Dhan background. The transgenic and control lines were exposed to 50 nM cadmium for 10 days. Data for the roots and shoots are mean±SD (n=4).

As shown in FIGS. 13 and 14, there was no difference in the concentrations of other micronutrients and macronutrients in both the roots and shoots between the control and transgenic lines.

These results demonstrated that OsHMA3 gene is a gene responsible for differential cadmium accumulation between Anjana Dhan and Nipponbare cultivars.

(4. Functional Analysis of RNAi Lines)

The accumulation of cadmium in RNAi lines of Nipponbare in which OsHMA3 gene expression had been knocked down by RNAi was examined.

For the purpose of generating a hairpin RNAi construct, a 511-bp fragment (893 bp to 1407 bp from the transcriptional start) of OsHMA3n cDNA was cloned as inverted repeats into a pANDA vector under the control of a maize ubiquitin promoter. For the purpose of generating a construct carrying a ubiquitin promoter, OsHMA3n gene and a NOS terminator were amplified from OsHMA3n cDNA by PCR using a primer set 5′-AGGATCCATGGCCGGAAAGGATGAGG-3′ (SEQ ID NO: 9) and 5′-TGGATCCGCAACATCATCCTTTCACTTCACC-3′ (SEQ ID NO: 10). The amplified fragment was cloned into a pANDA vector, excised together with a maize ubiquitin promoter, and then subcloned into a pPZP2H-lac binary vector.

The resulting constructs thus generated were introduced by an Agrobacterium method into rice calluses derived from Nipponbare.

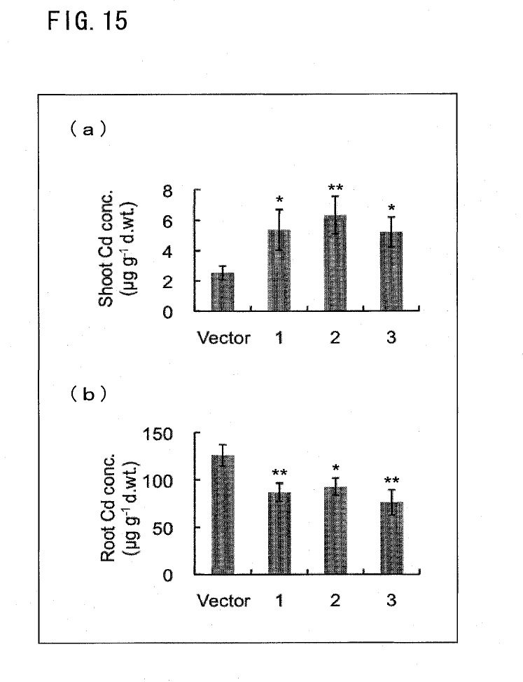

FIG. 15 shows results of measurement of levels of cadmium accumulation in RNAi lines of Nipponbare in which OsHMA3 gene expression was knocked down by RNAi and a control line carrying an empty vector in its Nipponbare background, (a) and (b) showing cadmium concentration in the shoots and roots, respectively, of the three RNAi lines and the control line. The RNAi and control lines were exposed to 50 nM cadmium for 10 days. Data for (a) and (b) are mean±SD (n=3). A statistical analysis was carried out according to Dunnett's t-test. In the graph, the asterisk “*” means that there is a significant difference between the control and RNAi lines at a significance level of less than 5%, and the double asterisk “**” means that there is a significant difference between the control and RNAi lines at a significance level of less than 1%.

As shown in (a) of FIG. 15, when OsHMA3 gene expression was knocked down by RNAi, the concentrations of cadmium in the shoots were significantly increased by 2.1 to 2.5 times in the RNAi lines as compared with the control line (p<0.05) and the concentrations of cadmium in the roots were significantly decreased by 74 to 60% in the RNAi line as compared with the control line (p<0.05).

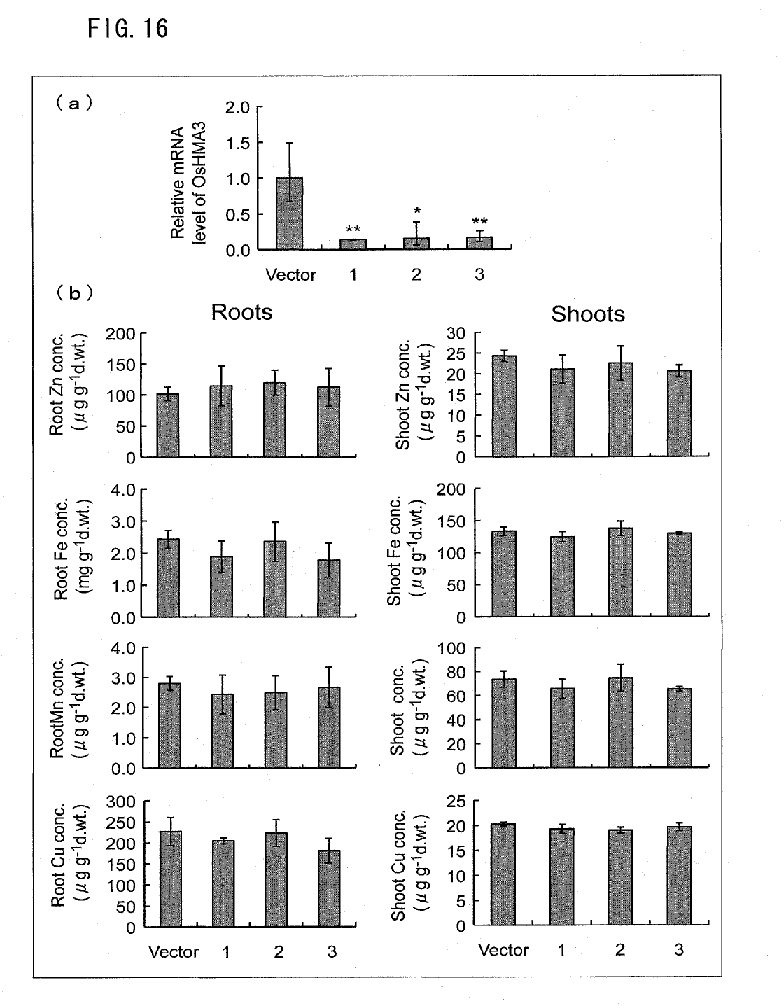

FIG. 16 shows concentrations of micronutrients in the RNAi lines of Nipponbare in which OsHMA3 gene expression was knocked down by RNAi and the control line carrying an empty vector in its Nipponbare background, (a) showing levels of OsHMA3 gene expression in the three RNAi lines and the control line, (b) showing concentrations of micronutrients in the shoots and roots of the RNAi and control lines.

The RNAi and control lines were exposed to 50 nM cadmium for 10 days. (a) of FIG. 16 shows the levels of OsHMA3 gene expression in the RNAi lines relative to the level of OsHMA3 gene expression in the control line.

Data for (a) and (b) are mean±SD (n=3). A statistical analysis was carried out according to Dunnett's t-test. In the graph, the asterisk “*” means that there is a significant difference between the control and RNAi lines at a significance level of less than 5%, and the double asterisk “**” means that there is a significant difference between the control and RNAi lines at a significance level of less than 1%.

As shown in (a) of FIG. 16, the RNAi lines showed significantly decreased levels of OsHMA3 gene expression as compared with the control line. Further, as shown in (b) of FIG. 16, there was no difference in the concentration of other micronutrients, including Zn, Cu, Mn, and Fe, in both the roots and the shoot between the RNAi and control lines.

These results further confirm that OsHMA3 gene is a gene responsible for differential cadmium accumulation observed in the two cultivars, Nipponbare and Anjana Dhan.

(5. Expression Pattern Analysis of OsHMA3 Genes)

The expression level of two allelic genes in different tissues of the two cultivars was determined by using quantitative real time RT-PCT.

The expression level analysis was carried out by Thunderbird™ qPCR Mix (manufactured by Toyobo) with use of the following primer pairs:

5′-TCCATCCAACCAAACCCGGAAA-3′ (SEQ ID NO: 11) and 5′-TGCCAATGTCCTTCTGTTCCCA-3′ (SEQ ID NO: 12) for use in OsHMA3 analysis; and

5′-GGTCAACTTGTTGATTCCCCTCT-3′ (SEQ ID NO: 13) and 5′-AACCGCAAAATCCAAAGAACG-3′ (SEQ ID NO: 14) for use in histone H3 analysis as an internal standard.

Data were collected in accordance with a 7500 Real Time PCR System (manufactured by Applied Biosystems). A ΔCt method was used to evaluate the relative quantities of each amplification product. Amplification efficiency of the real time PCR was checked by a standard curve obtained by using diluted plasmid DNA as a template. The amplification efficiency was 1.96 for both OsHMA3n and OsHMA3a genes.

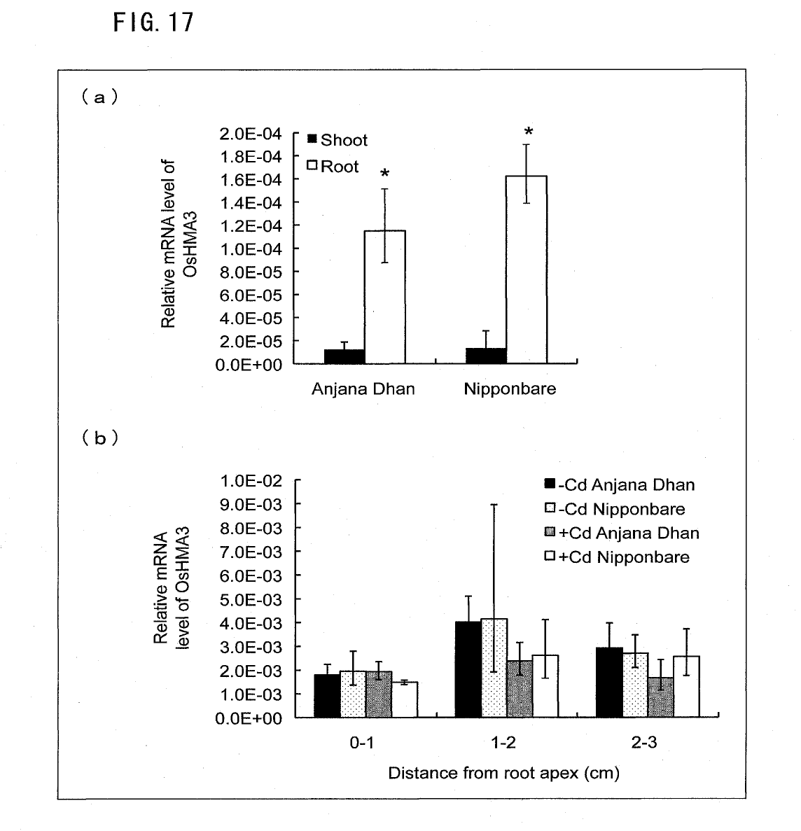

FIG. 17 shows an expression pattern of OsHMA3 genes. (a) of FIG. 17 shows the expression of two allelic genes of OsHMA3 in the roots and shoots of Nipponbare and Anjana Dhan. (b) of FIG. 17 shows the expression of two allelic genes in different parts of the roots of Nipponbare and Anjana Dhan.

For the purpose of investigating the expression pattern of OsHMA3 genes, RNA was extracted from the shoots and roots of Nipponbare and Anjana Dhan (each 12 days old). Specifically, RNA was extracted by excising the roots at different segments (0 to 1 cm, 1 to 2 cm, and 2 to 3 cm) of Nipponbare and Anjana Dhan seedlings exposed to 0 μM CdSO₄ or 1 μM CdSO₄ for 24 hours. Expression levels were determined by quantitative real time RT-PCT. Histone H3 was used as an internal standard. The levels of OsHMA3 expression are relative to the level of histone H3 expression. Data are mean±SD (n=3). A statistical analysis was carried out according to Dunnett's t-test. In the graph, the asterisk “*” means that there is a significant difference between the control and RNAi lines at a significance level of less than 5%.

As shown in (a) of FIG. 17, OsHMA3 genes were expressed mainly in the roots at similar levels in the two lines contrasting in cadmium accumulation. Further, as shown in (b) of FIG. 17, spatial analysis shows that there is no difference in the expression of OsHMA3 genes among different root segments. Furthermore, it became clear that the OsHMA3 gene expression is not affected by cadmium exposure. This result indicates the constitutive expression of OsHMA3 genes in the two lines.

(6. Localization Analysis of OsHMA3 Protein)

The localization of OsHMA3 protein was investigated by immunostaining and by using promoter-GFP transgenic rice.

An antibody against OsHMA3 protein was obtained by immunizing a rabbit with a synthetic peptide at the 993rd to 1004th positions in the amino acid sequence of OsHMA3n protein (SEQ ID NO: 1). The antiserum thus obtained was purified through a peptide affinity column before use. Immunostaining of OsHMA3 protein in the roots of Nipponbare and Anjana Dhan (each 10 days old) was carried out. Fluorescence of a secondary antibody (Alexa Fluor 555 goat anti-rabbit IgG; Molecular Probes) was observed with a confocal laser scanning microscopy (LSM700; Carl Zeiss).

For the purpose of constructing a translational pOsHMA3-GFP fusion gene, 2 kb of upstream region (−34 bp to −2094 bp from the translational start codon) of OsHMA3 gene was amplified by PCR from Nipponbare genomic DNA by using a primer set 5′-ATCTAGAAGCATAAAAGAATAGAGCCGTGGAC-3′ (SEQ ID NO: 15) and 5′-ATCTAGAATGCAAGTGGGGATCAAGGA-3′ (SEQ ID NO: 16). The promoter was cloned into the XbaI site of GFP and NOS terminator in a pUC18 vector (TaKaRa). The construct carrying the pOsHMA3n-GFP and NOS terminator was subcloned into a pPZP2H-lac binary vector. The resulting constructs thus generated were introduced by an Agrobacterium method into rice calluses derived from Nipponbare. GFP signals were observed with a confocal laser scanning microscopy (LSM700; Carl Zeiss).

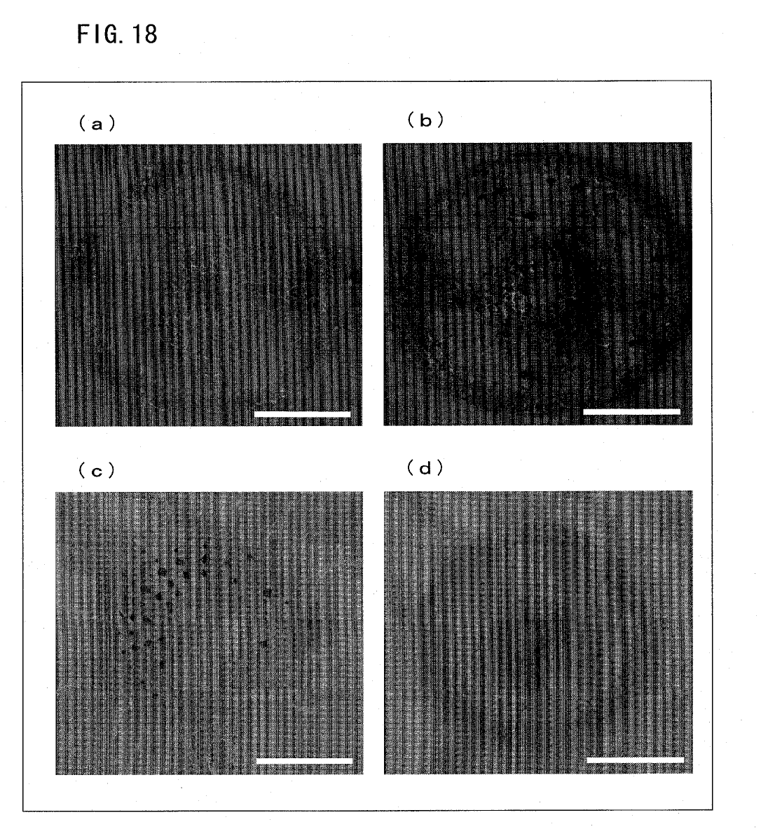

FIG. 18 shows the localization of OsHMA3 protein in rice roots. (a) of FIG. 18 shows a result of immunostaining in the roots of Anjana Dhan, and (b) of FIG. 18 shows a result of immunostaining in the roots of Nipponbare. In each of (a) and (b) of FIG. 18, the scale bar indicates 100 μm.

As shown in (a) and (b) of FIG. 18, immunostaining with anti-OsHMA3 polyclonal antibodies showed that OsHMA3 protein is localized in all root cells and there was no difference in the localization of OsHMA3 protein between Anjana Dhan and Nipponbare.

(c) of FIG. 18 shows fluorescence of GFP protein in the roots of pOsHMA3n-GFP transgenic Nipponbare, and (d) of FIG. 18 shows fluorescence of GFP protein in the roots of wild-type Nipponbare. In each of (c) and (d) of FIG. 18, the scale bar indicates 100

As shown in (c) of FIG. 18, pOsHMA3n-GFP transgenic Nipponbare, which carried GFP under the control of the OsHMA3n promoter, showed that OsHMA3n protein is expressed in all root cells. On the other hand, as shown in (d) of FIG. 18, no GFP signal was observed in the wild-type Nipponbare.

Although not illustrated, immunostaining of over-expression lines with anti-OsHMA3 antibodies showed greatly enhanced OsHMA3 protein signals. On the other hand, immunostaining of RNAi lines with anti-OsHMA3 antibodies showed very weak OsHMA3 protein signals. These results indicated the specificity of the anti-OsHMA3 antibodies used in immunostaining.

Although not illustrated, the OsHMA3 protein signals were each observed outside of the nucleus. This clearly showed that OsHMA3 protein is localized to the tonoplast.

The ORF of OsHMA3n gene and the OsHMA3a cDNA fragment were amplified by using a primer set 5′-ATCCGGAATGGCCGGAAAGGATGAGGC-3′ (SEQ ID NO: 17) and 5′-TTCCGGATCCTTTCACTTCACCGGAG-3′ (SEQ ID NO: 18). The OsHMA3 fragment was ligated to the 3′ end of GFP carrying a linker sequence (SGGGGGG) and placed under the control of a CaMV 35S promoter in pUC18 (TaKaRa). The resulting plasmid (pGFP-OsHMA3) or GFP alone was introduced into onion epidermal cells by particle bombardment (PDS-1000/He particle delivery system, Bio-Rad, http://www.bio-rad.com/) using 1100 psi pressure disks. GFP signals were observed with a confocal laser scanning microscopy (LSM700; Carl Zeiss).

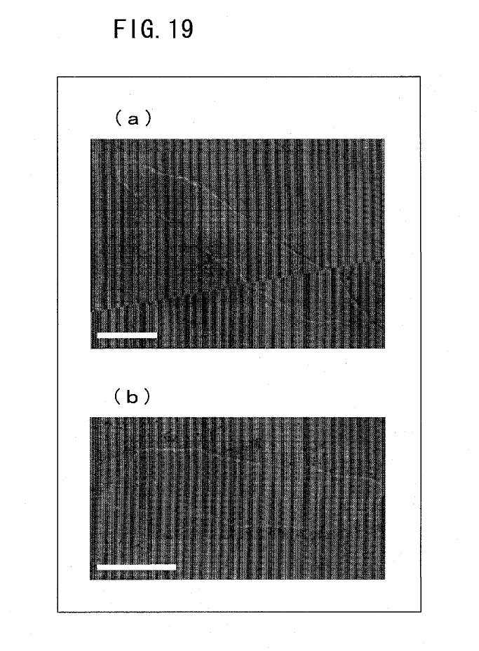

FIG. 19 shows subcellular localization of OsHMA3n protein. (a) of FIG. 19 shows fluorescence of GFP in onion epidermal cells expressing GFP-OsHMA3n protein, and (b) of FIG. 19 shows fluorescence of GFP in onion epidermal cells expressing GFP protein alone. In each of (a) and (b) of FIG. 19, the scale bar indicates 100 μm.

As shown in (a) of FIG. 19, transient expression of OsHMA3n-GFP fusion protein in onion epidermal cells clearly showed that OsHMA3n protein is localized to the tonoplast. Although not illustrated, a similar result was obtained for OsHMA3a protein. Furthermore, coexpression of GFP-OsHMA3n protein or GFP-OsHMA3a protein with DsRed-HDEL, which is an endoplasmic reticulum marker, further demonstrated the subcellular localization of OsHMA3 protein at the tonoplast, but not the endoplasmic reticulum (not illustrated).

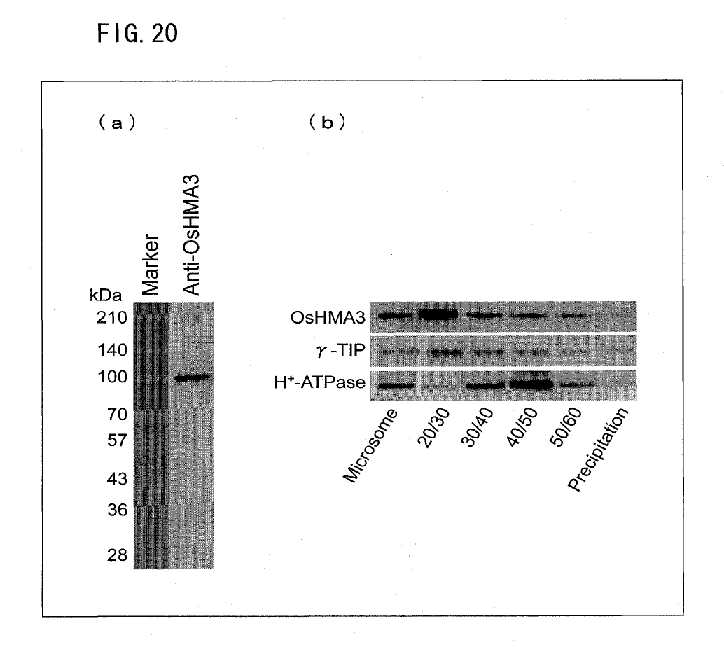

FIG. 20 shows results of Western blot analysis. Microsome extracted from the whole roots of OsHMA3n protein over-expression lines generated from Nipponbare (137 days old) was used for Western blot analysis with anti-OsHMA3n antibody. The microsome fraction was fractionated by sucrose-density gradient. As primary antibodies, anti-OsHMA3 polyclonal antibody (diluted hundredfold), anti-γ-TIP polyclonal antibody (tonoplast marker; diluted thousandfold), and anti-H+-ATPase polyclonal antibody (membrane marker) were used. As a secondary antibody, ELC peroxidase labelling anti-rabbit antibody (diluted ten-thousandfold; manufactured by GE Healthcare) was used. Signal detection was carried out by using an ECL Plus western blotting detection system (manufactured by GE Healthcare).

(a) of FIG. 20 shows the specificity of the anti-OsHMA3 antibody, and (b) of FIG. 20 shows results of sucrose-density gradient analysis.

As shown in (a) of FIG. 20, the Western blot analysis with the antibody against OsHMA3 protein showed a single band at the predicted size. This indicates the specificity of this antibody. Further, as shown in (b) of FIG. 20, the sucrose-density gradient analysis showed that OsHMA3 protein was present in the same fraction as γ-TIP, which is a tonoplast marker. Taken together, all these results indicate that OsHMA3 protein is localized to the tonoplast of rice root cells.

(6. Analysis of Transport Activity in Yeast)

For the purpose of understanding the large difference in cadmium accumulation between Anjana Dhan and Nipponbare, each gene was expressed in yeast.

Saccharomyces cerevisiae strain BY4741 (Mat a; his3Δ1; leu2Δ0; met15Δ0; ura3Δ0) and mutant strains Δzrc1 (Mat a; his3Δ1; leu2Δ0; met15Δ0; ura3Δ0; YMR243c::kanMX4) and Δcot1 (Mat a; his3Δ1; leu2Δ0, met15Δ0; ura3Δ0; YOR316c::kanMX4) were purchased from Euroscarf (http://web.uni-frankfurt.de/fb15/mikro/euroscarf/index.html).

OsHMA3a gene and OsHMA3n gene were amplified by PCR. In each case, a fragment containing the ORF was inserted into pYES2, which is a yeast expression vector.

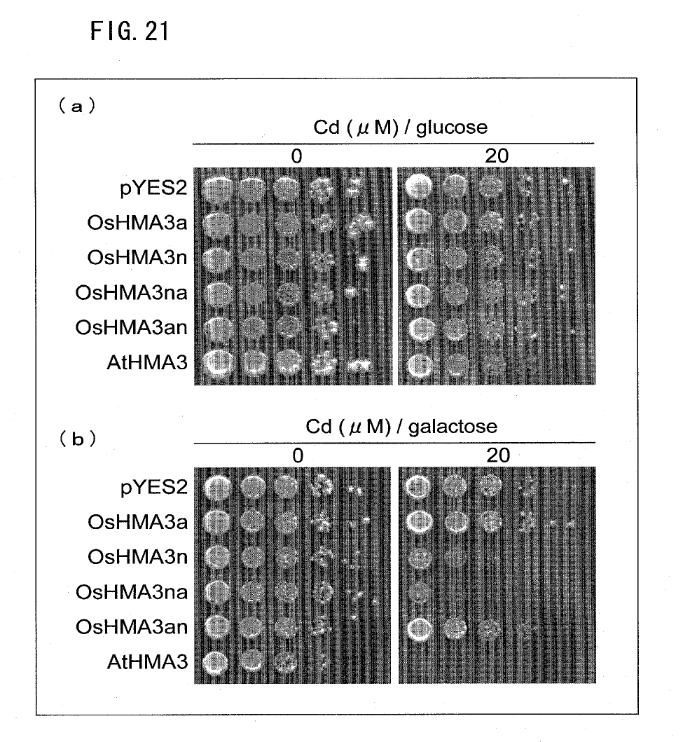

FIG. 21 shows results of functional analysis in yeast for cadmium. (a) of FIG. 21 shows the growth of wild-type yeast cells (BY4741 strain) transformed with a pYES2 vector alone, OsHMA3n gene, OsHMA3a gene, AtHMA3 gene, and two chimeric genes, respectively, where these wild-type yeast cells were grown in the presence of glucose, and (b) of FIG. 21 shows the growth of wild-type yeast cells (BY4741 strain) transformed with a pYES2 vector alone, OsHMA3n gene, OsHMA3a gene, AtHMA3 gene, and two chimeric genes, respectively, where these wild-type yeast cells were grown in the presence of galactose. In each case, the yeast was cultured in the presence of 20 μm CdSO₄.

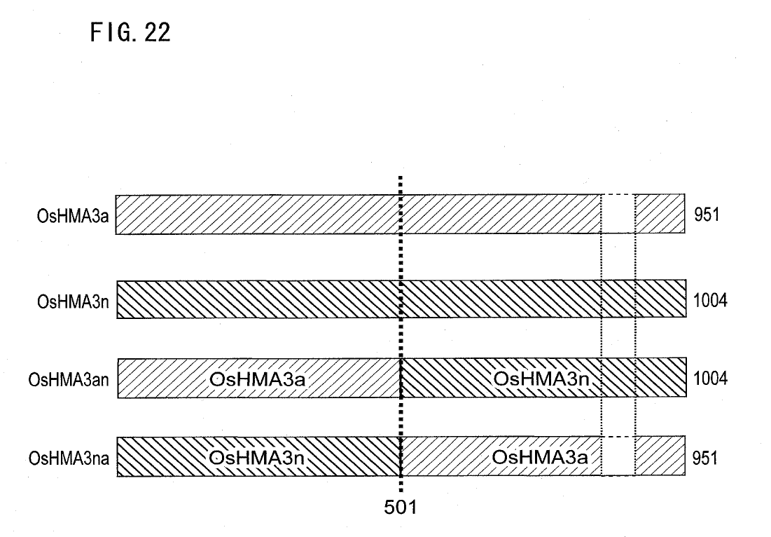

It should be noted that N-OsHMA3n-C-OsHMA3a(OsHMA3na) and N-OsHMA3a-C-OsHMA3n(OsHMA3an) are transgenic lines each expressing a chimera protein (see FIG. 22) obtained by fusing OsHMA3n protein and OsHMA3a protein at the 501st position in the amino acid sequence (SEQ ID NO: 1 or SEQ ID NO:2) of OsHMA protein.

501

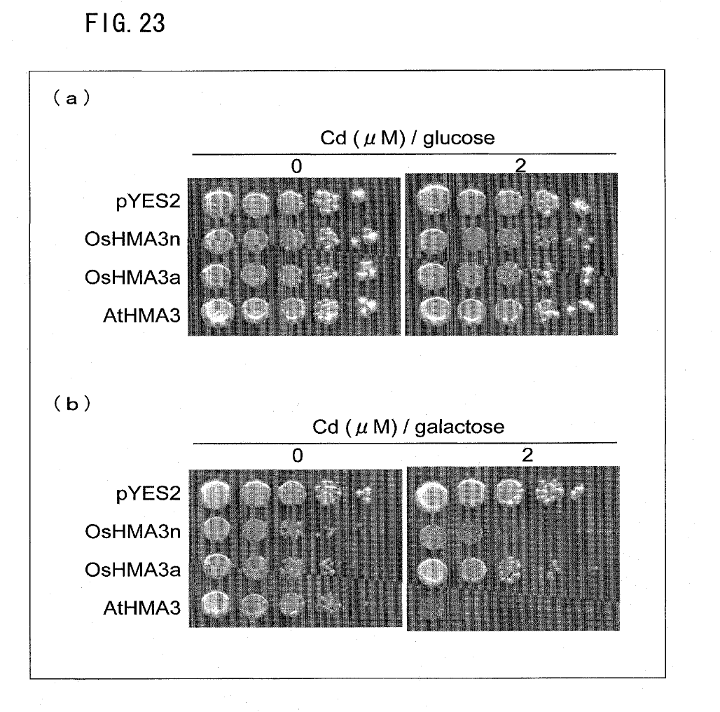

FIG. 23 shows results of functional analysis in yeast for cadmium. (a) of FIG. 12 shows the growth of Δycf1 yeast cells transformed with a pYES2 vector alone, OsHMA3n gene, OsHMA3a gene, and AtHMA3 gene, respectively, where these Δycf1 yeast cells were grown in the presence of glucose, and (b) of FIG. 12 shows the growth of Δycf1 yeast cells transformed with a pYES2 vector alone, OsHMA3n gene, OsHMA3a gene, and AtHMA3 gene, respectively, where these Δycf1 yeast cells were grown in the presence of galactose. In each case, the yeast was cultured for 3 days in a medium containing 2 μm cadmium.

Gene expression from a GAL1 promoter in a pYES2 vector is induced in the presence of galactose, but down-regulated in the presence of glucose. For this reason, glucose was added to the medium to down-regulate the expression of the transforming genes, and galactose was added to the medium to induce the expression of the transforming genes.

As shown in (a) of FIG. 21, in the presence of glucose, there was no difference in cadmium sensitivity between the yeast carrying a plasmid containing OsHMA3a gene and the yeast carrying a plasmid containing OsHMA3n gene. However, as shown in (b) of FIG. 21, in the presence of galactose, the yeast expressing OsHMA3n gene showed increased sensitivity to cadmium, but the yeast expressing OsHMA3a gene showed similar growth to the control.

Further, as shown in (a) and (b) of FIG. 23, expression of OsHMA3a and OsHMA3n genes in Δycf1 yeast, which is a cadmium sensitive mutant, also yielded the same results as those shown in FIG. 21. These results are consistent with OsHMA3n gene acting as a cadmium transporter in yeast, whereas OsHMA3a gene appears not to act as a cadmium transporter.

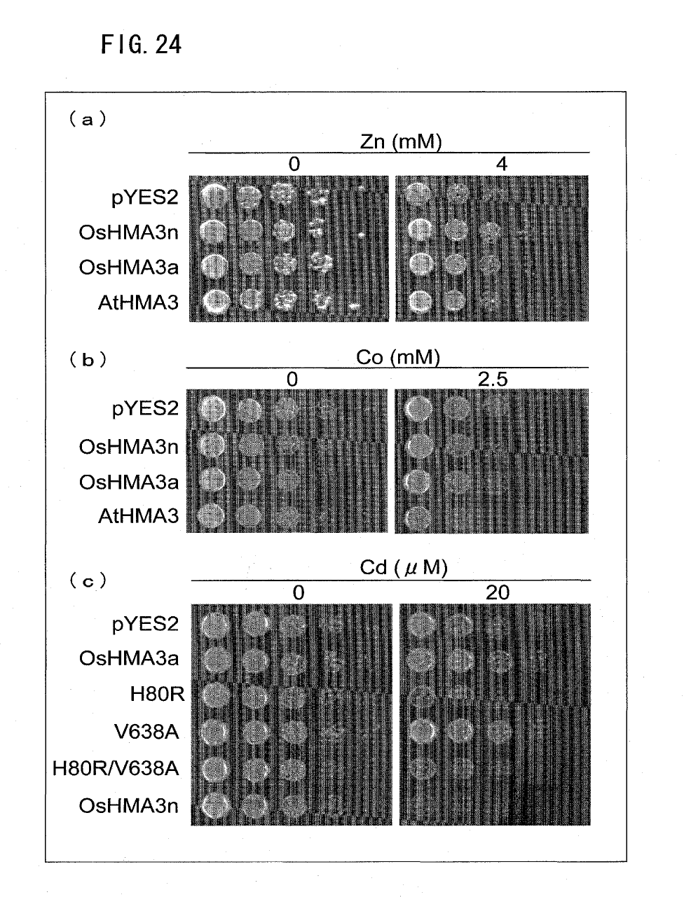

FIG. 24 shows results of functional analysis in yeast for metals. (a) of FIG. 24 shows the growth of Δzrc1 yeast strains transformed with a pYES2 vector alone, OsHMA3n gene, OsHMA3a gene, and AtHMA3 gene as a positive control, respectively, where these Δzrc1 yeast strains were grown in the presence of galactose and 4 mM ZnSO₄. (b) of FIG. 24 shows the growth of Δcot1 yeast strains transformed with a pYES2 vector alone, OsHMA3n gene, OsHMA3a gene, and AtHMA3 gene as a positive control, respectively, where these Δcot1 yeast strains were grown in the presence of galactose and 2.5 mM CoCl₂. (c) of FIG. 24 shows the growth of wild-type yeast cells transformed with a pYES2 vector alone, OsHMA3a gene, OsHMA3aH80R gene, OsHMA3aV638A gene, OsHMA3aH80R/V638A gene, and OsHMA3n gene, respectively, where these wild-type yeast strains were grown in the presence of galactose and 20 μm CdSO₄. All of the yeast strains were cultured at 30° C. for 3 days.

It should be noted that OsHMA3aH80R, OsHMA3aV638A, and OsHMA3aH80R/V638A are site-directed mutagenesized genes at the 80th and/or 638th position(s) in the base sequence of OsHMA3a gene (SEQ ID NO: 4).

As shown in (a) of FIG. 24, there was no difference in zinc sensitivity between the Δzrc1 yeast strain transformed with OsHMA3n gene and the Δzrc1 yeast strain transformed with OsHMA3a gene. Further, as shown in (b) of FIG. 24, there was no difference in cobalt sensitivity between the Δcot1 yeast strain transformed with OsHMA3n gene and the Δcot1 yeast strain transformed with OsHMA3a gene. On the other hand, the Δcot1 yeast strain transformed with AtHMA3 gene as a positive control showed increased sensitivity to cobalt.

OsHMA3 protein is localized at the tonoplast in rice roots ((a) and (b) of FIG. 19). If the localization of OsHMA3 protein is similar in yeast, expression of functional OsHMA3 protein should increase the tolerance to cadmium. However, the yeast expressing OsHMA3n gene showed increased cadmium sensitivity ((b) of FIG. 21). This discrepancy was considered to be attributed to the mislocalization of OsHMA3 protein in the yeast. Specifically, OsHMA3 protein was considered to be localized to the endoplasmic reticulum in yeast (not illustrated). Therefore, the functional OsHMA3 transports cadmium into the endoplasmic reticulum, thus resulting in increased cadmium sensitivity ((b) of FIG. 21).

For the purpose of dissecting a mechanism underlying the loss of function of OsHMA3a gene, two chimera proteins between OsHMA3a protein and OsHMA3n protein were prepared. The most different part between OsHMA3a protein and OsHMA3n protein lies in the C terminus. That is, in OsHMA3a protein, 53 amino acid residues are missing within the putative metal binding domain repeat (nine repeats in OsHMA3n protein and six repeats in OsHMA3a protein) (see FIG. 6).

The role of the putative metal binding domain repeat in transport activity was examined by using a chimera protein obtained by fusing the N terminus from OsHMA3a protein with the C terminus of OsHMA3n protein at the 501st position in the amino acid sequence (SEQ ID NO: 2) of OsHMA3a protein and a chimera protein obtained by fusing the N terminus from OsHMA3n protein with the C terminus of OsHMA3a protein at the 501st position in the amino acid sequence (SEQ ID NO: 1) of OsHMA3n protein (see FIG. 22).

In the N-OsHMA3n-C-OsHMA3a chimera protein, the cadmium sensitivity was increased as observed in OsHMA3n protein ((b) of FIG. 21). However, the N-OsHMA3a-C-OsHMA3n chimera protein did not change the cadmium sensitivity ((b) of FIG. 21). These results indicate that the N-terminal region in OsHMA3n protein, not the missing 53 residues at the C terminus of OsHMA3a protein, is important for the cadmium transport function.

Then, the N-terminal parts of two allelic genes were further compared. With use of a transmembrane domain prediction program (SOSUI; http://bp.nuap.nagoya-u.ac.jp/sosui/), it was found that mutations of amino acids at the 80th and 638th positions in the amino sequence (SEQ ID NO: 2) of OsHMA3a protein result in change of predicted transmembrane domain numbers. In order to determine whether these mutations are responsible for the loss of function of OsHMA3a protein, site-directed mutagenesis analysis was carried out by using a yeast expression system.

As shown in (c) of FIG. 24, when histidine at the 80th position in the amino acid sequence (SEQ ID NO: 2) of OsHMA3a protein was substituted by arginine (H80R), enhanced sensitivity to cadmium was observed. However, substitution of valine at the 638th position by alanine (V638A) did not effect a change in cadmium sensitivity (FIG. 3E). This result is consistent with that of the chimera experiment (8b) of FIG. 23). Substitution of amino acids at the 80th and 638th positions (H80R/V638A) also gave increased sensitivity to cadmium ((c) of FIG. 24). These results indicate that the amino acid at the 80th position in the amino acid sequence (SEQ ID NO: 1) of OsHMA3n protein might be critical for the function of OsHMA3n protein.

(7. Functional Analysis of Transgenic Lines 2)

For the purpose of testing the possibility that the accumulation of cadmium in the grains could be reduced by manipulating the OsHMA3 gene expression level, OsHMA3n gene was overexpressed in Nipponbare, which is a low cadmium-accumulating line, under the control of a maize ubiquitin promoter.

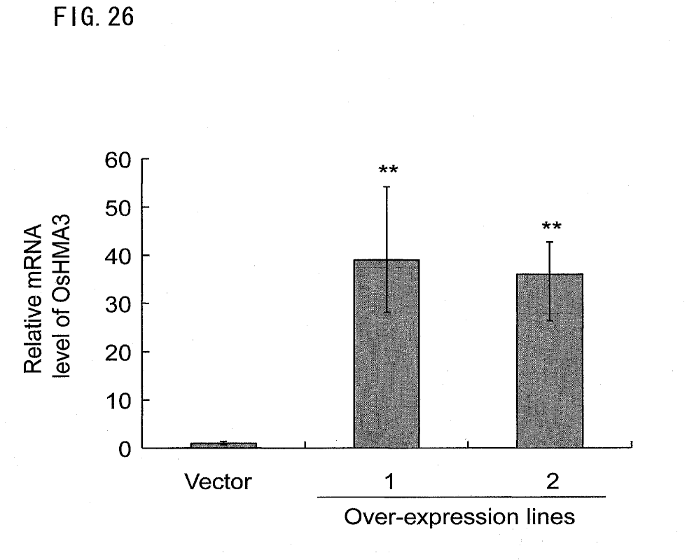

FIG. 26 shows levels of OsHMA3n gene expression in over-expression lines and in a vector control line. The levels of expression in the over-expression lines are relative to the level of expression in the vector control. Data are mean±SD (n=3). Further, statistical analysis was carried out according to Dunnett's t-test. In the graph, the double asterisk “*” means that there is a significant difference between the vector control and over-expression lines at a significance level of less than 1%.

As shown in FIG. 26, the over-expression lines showed significantly increased OsHMA3 gene expression as compared with the vector control line.

The over-expression lines, the vector control, and a nontransgenic line of Nipponbare were grown in a moderately cadmium-contaminated soil (1.5 mgCd kg⁻¹) without flooding for 5 months. After the treatment, the shoots, including leaf blades and leaf sheaths, the roots, and the brown rice were harvested for determination of cadmium and other metals by flame atomic absorption spectrometry (Z-2000; manufactured by Hitachi).

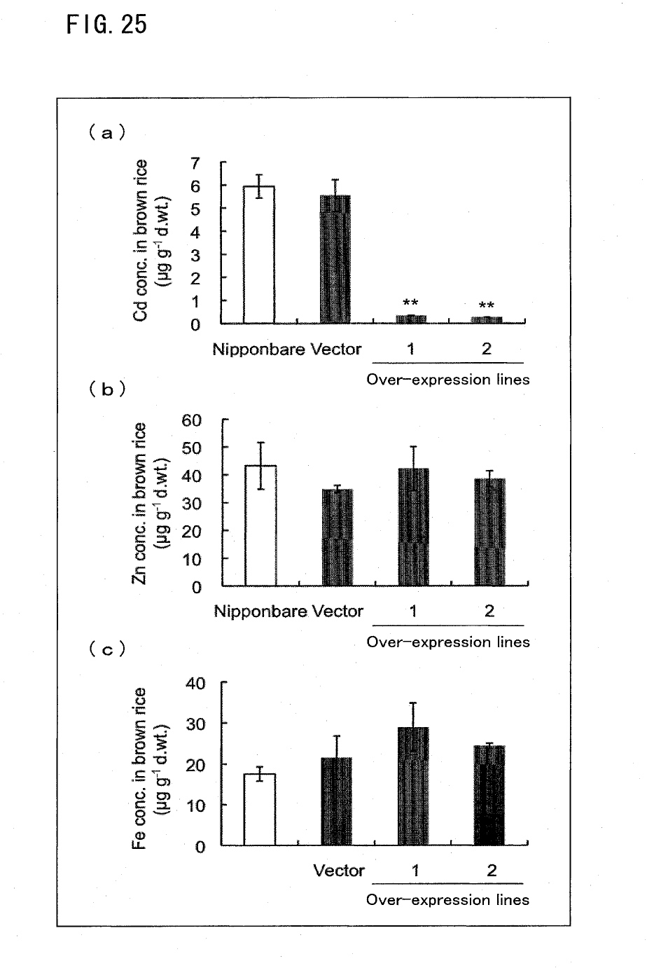

The results are shown in FIG. 25. FIG. 25 shows an effect of OsHMA3n gene overexpression on the accumulation of cadmium and on the accumulation of other metals, (a), (b), and (c) showing the concentrations of cadmium, zinc, and iron in the brown rice, respectively. Data are mean±SD (n=3). Further, statistical analysis was carried out according to Dunnett's t-test. In the graph, the double asterisk “**” means that there is a significant difference between the vector control and over-expression lines at a significance level of less than 1%.

As shown in (a) of FIG. 25, when grown in a cadmium-contaminated soil, the over-expression lines showed significantly lower cadmium concentration in the brown rice than the vector control line and the nontransgenic line (p<0.01). Further, as shown in (b) and (c) of FIG. 25, there was no difference in the concentrations of zinc and iron in the brown rice between the vector control line and the over-expression lines.

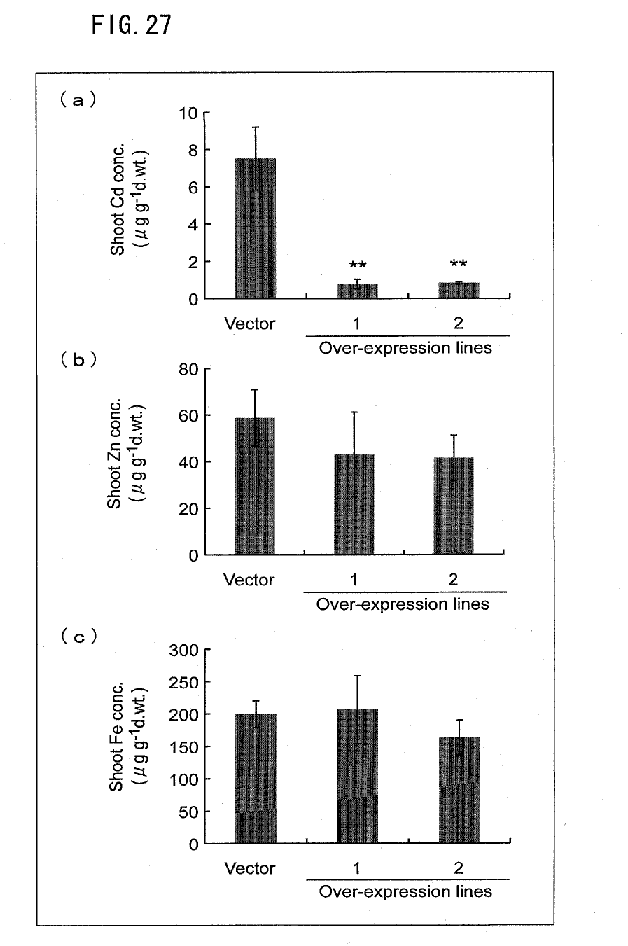

FIG. 27 shows an effect of OsHMA3n gene overexpression on the accumulation of cadmium and on the accumulation of other metals, (a), (b), and (c) showing the concentrations of cadmium, zinc, and iron in the shoots, respectively. Data are mean±SD (n=3). Further, statistical analysis was carried out according to Dunnett's t-test. In the graph, the double asterisk “*” means that there is a significant difference between the vector control and over-expression lines at a significance level of less than 1%.

As shown in (a) to (c) of FIG. 27, a similar trend was observed in the shoots to that observed in the brown rice.

### INDUSTRIAL APPLICABILITY

The present invention makes it possible to produce a transgenic plant changed in localization of cadmium accumulation. Such a transgenic plant can be used for various purposes. For example, even a cadmium-containing soil can be cultivated with a transgenic plant with such a change in localization of cadmium accumulation that cadmium accumulation is more localized in the roots. Further, a transgenic plant with such a change in localization of cadmium accumulation that cadmium accumulation is more localized in those parts of the transgenic plant except for the roots can be used to perform decontamination for a cadmium-containing soil. Therefore, the present invention can be suitably applicable to agriculture.

1. A method for reducing or increasing cadmium accumulation in those parts of a plant except for roots, comprising the step of, in reducing cadmium accumulation in those parts of the plant except for the roots, introducing a polynucleotide (a) or (b) into the plant such that the polynucleotide (a) or (b) is able to be expressed or, in increasing cadmium accumulation in those parts of the plant except for the roots, introducing a polynucleotide (c) or (d) into the plant such that the polynucleotide (c) or (d) is able to be expressed,
the method specifically reducing or increasing only cadmium accumulation in those parts of the plant except for the roots without affecting transport of metals other than cadmium, macronutrients, and micronutrients in the plant,
the polynucleotide (a) being
a polynucleotide encoding a polypeptide consisting of the amino acid sequence of SEQ ID NO: 1,
the polynucleotide (b) being a polynucleotide encoding a polypeptide in which one or several amino acids are substituted, deleted, and/or added in the amino acid sequence of SEQ ID NO: 1 and which has activity to specifically reduce only cadmium accumulation in those parts of the plant except for the roots without affecting the transport of metals other than cadmium, macronutrients, and micronutrients,
the polynucleotide (c) being a polynucleotide encoding a polypeptide consisting of the amino acid sequence of SEQ ID NO: 2,
the polynucleotide (d) being a polynucleotide encoding a polypeptide in which one or several amino acids are substituted, deleted, and/or added in the amino acid sequence of SEQ ID NO: 2 which has activity to specifically increase only cadmium accumulation in those parts of the plant except for the roots without affecting the transport of metals other than cadmium, macronutrients, and micronutrients.

2. The method as set forth in claim 1, wherein the plant is a poaceous plant.

3. A transgenic plant those parts of which except for roots have been specifically reduced or increased only in cadmium accumulation by a method as set forth in claim 1 without affecting the transport of metals other than cadmium, macronutrients, and micronutrients.

4. A kit for implementing a method as set forth in claim 1, comprising
any one of the following polynucleotides (a) to (d):
(a) a polynucleotide encoding a polypeptide consisting of the amino acid sequence of SEQ ID NO: 1;
(b) a polynucleotide encoding a polypeptide in which one or several amino acids are substituted, deleted, and/or added in the amino acid sequence of SEQ ID NO: 1 and which has activity to specifically reduce only cadmium accumulation in those parts of the plant except for the roots without affecting the transport of metals other than cadmium, macronutrients, and micronutrients;
(c) a polynucleotide encoding a polypeptide consisting of the amino acid sequence of SEQ ID NO: 2; or
(d) a polynucleotide encoding a polypeptide in which one or several amino acids are substituted, deleted, and/or added in the amino acid sequence of SEQ ID NO: 2 and which has activity to specifically increase only cadmium accumulation in those parts of the plant except for the roots without affecting the transport of metals other than cadmium, macronutrients, and micronutrients.
